(19) United States
(12) Patent Application Publication
(10) Pub. No.: US 2025/0210132 A1
(43) Pub. Date: Jun. 26, 2025

(54) LOW-COVERAGE, GENOME-WIDE IDENTIFICATION OF MINORITY cfDNA CONTRIBUTORS

(71) Applicant: Nucleix Ltd., Rehovot (IL)

(72) Inventors: Mathias EHRICH, San Diego, CA (US); Christopher ELLISON, San Diego, CA (US); Dirk VAN DEN BOOM, Encinitas, CA (US)

(21) Appl. No.: 18/793,536

(22) Filed: Aug. 2, 2024

Related U.S. Application Data
(60) Provisional application No. 63/517,741, filed on Aug. 4, 2023.

(51) Int. Cl. G16B 20/20; C12Q 1/6806; C12Q 1/6869; G16B 30/10; G16H 50/20; G16H 50/30

(52) U.S. Cl. CPC G16B 20/20; C12Q 1/6806; C12Q 1/6869; G16B 30/10; G16H 50/20; G16H 50/30

(57) ABSTRACT

In some aspects, the present disclosure provides a method for analyzing cell free DNA (cfDNA). The method can comprise obtaining a biological sample derived from a subject, wherein the biological sample comprises cfDNA. The method can comprise enriching a proportion of cfDNA within the biological sample. The method can comprise sequencing the cfDNA enriched biological sample using low-coverage, genome-wide nucleic acid sequencing. The method can comprise identifying a plurality of minority components present in the sequenced cfDNA enriched biological sample. The method can comprise assigning a designation that represents a low-confidence estimate of minor variant frequency to individual identified minority components present in the sequenced cfDNA enriched biological sample. The method can comprise averaging a plurality of low-confidence estimates of minor variant frequency across a plurality of sequenced genomic loci to produce an estimation of minority component frequency in the cfDNA enriched biological sample.

### CROSS-REFERENCE

This application claims the benefit of U.S. Provisional Application No. 63/517,741, filed on Aug. 4, 2023, which is incorporated herein by reference in its entirety.

### TECHNICAL FIELD

In some aspects described herein are methods for analyzing cell free DNA (cfDNA) utilizing low-coverage, genome-wide analysis to detect and identify minority cfDNA contributors in a biological sample. In some aspects described herein are kits for analyzing cell free DNA (cfDNA) providing instructions for utilizing low-coverage, genome-wide analysis to detect and identify minority cfDNA contributors in a biological sample

### BACKGROUND

Minority components in biological samples can provide early diagnosis of transplant rejection or transplant failure events and cancer remission or cancer progression.

### SUMMARY

In some aspects described herein are methods for analyzing cell free DNA (cfDNA), the methods comprising: obtaining a biological sample derived from a subject, wherein the biological sample comprises cfDNA, enriching a proportion of cfDNA within the biological sample, sequencing the cfDNA enriched biological sample using low-coverage, genome-wide nucleic acid sequencing, identifying a plurality of minority components present in the sequenced cfDNA enriched biological sample, assigning a designation that represents a low-confidence estimate of minor variant frequency to individual identified minority components present in the sequenced cfDNA enriched biological sample, and averaging a plurality of low-confidence estimates of minor variant frequency across a plurality of sequenced genomic loci to produce an estimation of minority component frequency in the cfDNA enriched biological sample. In some embodiments, assigned designation is a binary classifier for the individual identified minority components to distinguish a plurality of sequenced genomic loci from sequenced genomic loci not identified as having a minor variant in the sequenced cfDNA enriched biological sample. In some embodiments, identifying a plurality of minority components present in the sequenced cfDNA enriched biological sample comprises: aligning raw sequence data generated with low-coverage whole genome sequencing (lcWGS) to a reference sequence, marking duplicate reads of sequenced fragments, conducting pre-processing of BAM files generated following lcWGS by base quality score recalibration (BQSR), performing local realignment of sequences from pre-processed BAM files to produce analysis-ready BAM files, and performing variant calling on analysis-ready BAM files to identify the minority components. In some embodiments, a reference sample comprising genomic DNA derived from the subject is analyzed to distinguish somatic genotypes present in the subject from identified minority components in the cfDNA enriched biological sample. In some embodiments, the somatic genotypes are identified through high confidence genotyping. In some embodiments, the high confidence genotyping comprises sequencing of at least 2×, 3×, 4×, 5×, 6×, 7×, 8×, 9×, 10×, 12×, 15×, or 20× genomic coverage. In some embodiments, the sites sequenced with low-coverage, genome-wide nucleic acid sequencing are agnostic to pre-defined genomic loci. In some embodiments, the estimation of minority component frequency in the cfDNA enriched biological sample is a quantitative detection of a minority component present in the cfDNA of the biological sample. In some embodiments, the detected minority component present in the cfDNA of the biological sample comprises less than 25%, 20%, 15%, 12.5%, 10%, 9%, 8%, 7%, 6%, 5%, 4%, 3%, 2%, 1.5%, 1.4%, 1.3%, 1.25%, 1.2%, 1.15%, 1.1%, 1.05%, 1.0%, 0.95%, 0.9%, 0.8%, 0.7%, 0.6%, 0.5%, 0.45%, 0.4%, 0.35%, 0.3%, 0.25%, 0.2%, 0.15%, 0.1%, 0.09%, 0.08%, 0.07%, 0.06%, or 0.05% of the total cfDNA present in the biological sample. In some embodiments, a number of genomic loci assayed by the low-coverage, genome-wide nucleic acid sequencing to detect potential variants is at least 5000, 10000, 20000, 35000, 50000, 75000, 100000, 150000, 200000, 250000, 300000, 400000, 500000, 600000, 750000, 875000, 1000000, 2000000, 3000000, 4000000, 5000000, 7500000, or 10000000 genomic loci. In some embodiments, the biological sample is from serum, plasma, blood, saliva, urine, mucus, tears, sweat, semen, breast milk, lymphatic fluid, cerebrospinal fluid, or amniotic fluid of the subject. In some embodiments, the biological sample is from plasma of the subject. In some embodiments, the volume of the biological sample obtained from the subject is less than about 200 μL, 150 μL, 125 μL, 100 μL, 80 μL, 75 μL, 70 μL, 60 μL, 55 μL, 50 μL, 45 μL, 40 μL, 35 μL, 30 μL, 25 μL, 20 μL, 17.5 μL, 15 μL, 12.5 μL, 10 μL, 9 μL, 8 μL, 7 μL, 6 μL, 5 μL, 4 μL, 3 μL, 2.5 μL, 2 μL, 1.5 μL, 1 μL, or 0.5 μL. In some embodiments, the volume of blood obtained from the subject for the biological sample is less than about 200 μL, 150 μL, 125 μL, 100 μL, 80 μL, 75 μL, 70 μL, 60 μL, 55 μL, 50 μL, 45 μL, 40 μL, 35 μL, 30 μL, 25 μL, 20 μL, 17.5 μL, 15 μL, 12.5 μL, 10 μL, 9 μL, 8 μL, 7 μL, 6 μL, 5 μL, 4 μL, 3 μL, 2.5 μL, 2 μL, 1.5 μL, 1 μL, or 0.5 μL. In some embodiments, the biological sample is obtained from the subject by a method using capillary-based collection. In some embodiments, the individual identified minority components in cfDNA in e) comprise alternate heterozygous alleles or alternate homozygous alleles when compared to the alleles of genomic DNA from the subject. In some embodiments, the individual identified minority components in cfDNA in e) comprise alternate homozygous alleles when compared to the alleles of genomic DNA from the subject. In some embodiments, the variants detected comprise single-nucleotide polymorphisms (SNPs), small insertions or deletions (INDELs), variable number of tandem repeats (VNTR), simple sequence repeats (SSR), or simple tandem repeats (STR), or any combination thereof. In some embodiments, the variants detected comprise SNPs. In some embodiments, the low-coverage, genome-wide nucleic acid sequencing comprises less than about 1×, 0.9×, 0.8×, 0.7×, 0.6×, 0.5×, 0.4×, 0.35×, 0.3×, 0.25×, 0.2×, 0.15×, 0.125×, 0.1×, 0.09×, 0.08×, 0.075×, 0.065×, 0.05×, 0.035×, 0.025×, 0.02×, 0.015×, 0.01×, or 0.005× sequencing coverage of the genome of the subject. In some embodiments, the identified minority component alternate homozygous alleles comprise less than about 1000, 750, 500, 400, 300, 200, 150, 125, 110, 100, 90, 80, 75, 70, 65, 60, 55, 50, 45, 40, 35, 30, 25, or 20 loci. In some embodiments, the identified minority component alternate homozygous alleles comprise less than about 1000, 750, 500, 400, 300, 200, 150, 125, 110, 100, 90, 80, 75, 70, 65, 60, 55, 50, 45, 40, 35, 30, 25, or 20 SNP loci. In some embodiments, the alternate heterozygous alleles or alternate homozygous alleles are derived from cfDNA from pre-malignant or malignant cells of the subject. In some embodiments, the alternate heterozygous alleles or alternate homozygous alleles are derived from cfDNA from one or more infectious agents residing within the subject. In some embodiments, the alternate heterozygous alleles or alternate homozygous alleles are derived from a donor subject. In some embodiments, the donor subject is an embryo. In some embodiments, the donor subject is a fetus. In some embodiments, the donor subject has provided a tissue or an organ transplant into the subject which serves as a host. In some embodiments, the estimation of minority component frequency in the cfDNA enriched biological sample is used for transplantation monitoring. In some embodiments, transplantation monitoring comprises distinguishing non-rejection (TX) from one or both of acute rejection (AR) and acute dysfunction non-rejection (ADNR). In some embodiments, transplantation monitoring comprises comparing a level of detected donor derived cell-free DNA (dd-cfDNA) in the cfDNA enriched biological sample to a pre-determined threshold value. In some embodiments, the host is indicated as having a likelihood of AR or ADNR by a level of dd-cfDNA that is greater than or equal to a pre-determined threshold value of at least 0.5%, 0.6%, 0.75%, 0.8%, 0.85%, 0.9%, 0.95%, 1.0%, 1.1%, 1.25%, 1.5%, 1.75%, 2.0%, 2.5%, 3.0%. 4.0%, 5.0%, 7.5%, or 10%. In some embodiments, the host is indicated as having a likelihood of non-rejection by a level of dd-cfDNA that is less than a pre-determined threshold of about 1.5%, 1.25%, 1.0%, 0.9%, 0.8%, 0.7%, 0.6%, 0.5%, 0.45%, 0.4%, 0.35%, 0.30%, 0.25%, 0.2%, 0.15%, or 0.1%. In some embodiments, an increasing presence of donor derived cell-free DNA over time indicates failure or rejection of a transplanted organ or tissue. In some embodiments, the estimation of minority component frequency in the cfDNA enriched biological sample is used for oncology detection or oncology monitoring. In some embodiments, at least two biological samples derived from the subject at different time points are analyzed to screen for a change in minor cfDNA components over time. In some embodiments, a significant increase in somatic variants over time is detected. In some embodiments, the method further comprises identification of new somatic variants detected in a biological sample collected from the subject after collection of an initial biological sample from the subject. In some embodiments, somatic SNPs are distinguished from germline SNPs in the subject. In some embodiments, an initial biological sample is obtained from a tumor biopsy. In some embodiments, the method further comprises analyzing patterns of DNA methylation in the cfDNA enriched biological sample. In some embodiments, the method further comprises analyzing a proportion of detected single-stranded cfDNA to detected double-stranded cfDNA in the cfDNA enriched biological sample. In some embodiments, the method comprises monitoring progression of an infectious disease. In some embodiments, the method comprises comparing a level of detected cfDNA from one or more infectious agents in the cfDNA enriched biological sample to a pre-determined threshold value. In some embodiments, monitoring progression of an infectious disease comprises detecting a significant increase in cfDNA from one or more infectious agents over time. In some embodiments, the subject is monitored for progression of sepsis. In some embodiments, a development or progression of pregnancy complications is monitored. In some embodiments, the method further comprises analyzing fragment patterning in the cfDNA enriched biological sample. In some embodiments, the method further comprises imputing missing SNP genotypes in the sequenced cfDNA enriched biological sample. In some embodiments, the method further comprises calculating and evaluating regional linkage disequilibrium ratios between variant alleles detected to enhance the calculated estimation of minority component frequency in the cfDNA enriched biological sample. In some embodiments, the method further comprises individual subject level tuning comprising longitudinal sampling of biological samples obtained from the subject to screen for change in minor cfDNA components over time. In some embodiments, the low-coverage, genome-wide nucleic acid sequencing is unbiased sequencing.

In some aspects described herein are kits for analyzing cell free DNA (cfDNA), the kits comprising: a sample collector configured to collect a biological sample of a subject; and a set of instructions for: enriching a proportion of cfDNA within the biological sample, sequencing the cfDNA enriched biological sample using low-coverage, genome-wide nucleic acid sequencing, identifying a plurality of minority components present in the sequenced cfDNA enriched biological sample, assigning a designation that represents a low-confidence estimate of minor variant frequency to individual identified minority components present in the sequenced cfDNA enriched biological sample, and averaging a plurality of low-confidence estimates of minor variant frequency across a plurality of sequenced genomic loci to produce an estimation of minority component frequency in the cfDNA enriched biological sample. In some embodiments, the sample collector is configured to collect one or more biological samples comprising whole blood from the subject.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an illustrative schematic of the method, in accordance with some embodiments.

FIG. 2A and FIG. 2B show limit of quantitation and limit of detection, in accordance with some embodiments. FIG. 2A shows a graph representing limits of quantitation relating to samples of varying concentrations of target donor-derived cell-free DNA (dd-cfDNA). FIG. 2B shows a graph representing limits of quantitation (LoQ), limits of detection (LoD), and limit of blank (LoB) relating to samples of varying concentrations of target dd-cfDNA.

FIG. 3 shows a computer system, in accordance with some embodiments.

### DETAILED DESCRIPTION

Quantitative identification of minority components that are present in cell-free nucleic acids, (e.g., cell-free DNA; cfDNA) can be used for various non-invasive diagnostic testing strategies. It can be used for transplantation monitoring, oncology detection and monitoring, prenatal testing, screening and detection of monogenic diseases, and infectious disease diagnosis and monitoring. For some applications, quantitative detection of low fractions—as low as 1%, 0.5%, or even 0.1% of the total cell-free nucleic acid material—of a minority component is desirable. In transplant monitoring, the presence of greater than a certain threshold value (e.g., about 1%) of cell-free nucleic acids derived from transplanted tissue can indicate active rejection or organ failure of the transplant tissue. The indication can serve as a basis for medical intervention. In oncological monitoring, tumor-derived cell-free nucleic acids can indicate cancer recurrence after surgery and/or chemotherapy. Furthermore, changes in the composition of cell-free nucleic acids over time can indicate the development or progression of certain diseases. As a non-limiting example, detectable or increasing concentration of one or more non-germline polymorphisms in cfDNA samples from a subject may indicate progression of a cancer wherein DNA repair mechanisms in tumor cells are impaired. As another non-limiting example, detectable or increasing concentration of one or more non-germline polymorphisms in cfDNA samples from a subject may indicate clonal expansion of pathological cells in a disorder (e.g., an autoimmune disorder, a disorder of pre-malignant transformation, malignant proliferation in a cancer such as a leukemia, or malignant proliferation and/or metastasis in a cancer such as a lymphoma, a carcinoma, or a sarcoma). Accordingly, close and frequent monitoring of cell-free nucleic acids is desirable to provide early intervention for individuals who may be affected by, e.g., transplant rejection or cancer recurrence. A non-invasive or a minimally invasive method for detecting minority components is particularly advantageous, because it reduces burden on the patients and medical professionals for obtaining workable samples. It can also permit patients to use at-home test kits to provide workable samples to laboratories. A non-invasive or a minimally invasive method for detecting minority components of cfDNA that is sensitive and accurate can provide clinically informative and diagnostic information at an early time point in progression of a condition or disease to allow i) earlier initiation of a treatment; ii) earlier modification of an ongoing treatment, iii) an earlier gating decision for treatment switching, or iv) any combination thereof, compared to a less sensitive or less accurate method of detecting minority components of cfDNA

Although it is desirable to detect low amounts of minority components in cell-free nucleic acids from biological samples with high confidence, the identification of minority components can be challenging when there are large amounts of nucleic acids derived from native or healthy cell populations (e.g., derived from cells of the host, non-cancer cells of a patient, or host cellular nucleic acid contamination in a cell-free nucleic acid biological sample). One approach to meet this challenge is via high depth sampling at a small number of genomic loci. The high depth can improve statistical certainty. Sequencing can be performed using techniques such as targeted deep sequencing, digital PCR, or allele-specific PCR. A focus of such approaches is to distinguish between low-frequency minority variants and majority background cfDNA. These methods can use tens to thousands of loci to estimate the relative frequency of the minor component.

In contrast, a genome-wide approach can be utilized to cover hundreds of thousands to millions of potentially informative loci. Low-confidence estimates of minor variant frequency across many loci can be averaged to provide an estimate of minority component frequency. This is distinguished from the high-depth approach of using high-confidence estimates of minor variant frequency across fewer loci. Because the genomic-wide approach does not require high-depth profiling, a genome-wide approach can be used to screen for minority components from less starting material (e.g. microliters/drops of blood versus milliliters/tubes of blood). In other words, a genome-wide approach can use very small samples which inherently do not contain sufficient cfDNA to provide high-depth profiling at a sufficient number of loci, or even provide a signal for pre-selected genomic loci. Furthermore, because the targeted approaches may add additional processing steps, a genome-wide approach can include additional nucleic acid diagnostic features (e.g., epigenetic marks, fragment size profiles, alignment biases, first base biases, etc.) that further refine the sensitivity of the approach. In some aspects, the present disclosure provides a low-coverage, genome-wide sequencing method for quantitatively assessing the abundance of minor components.

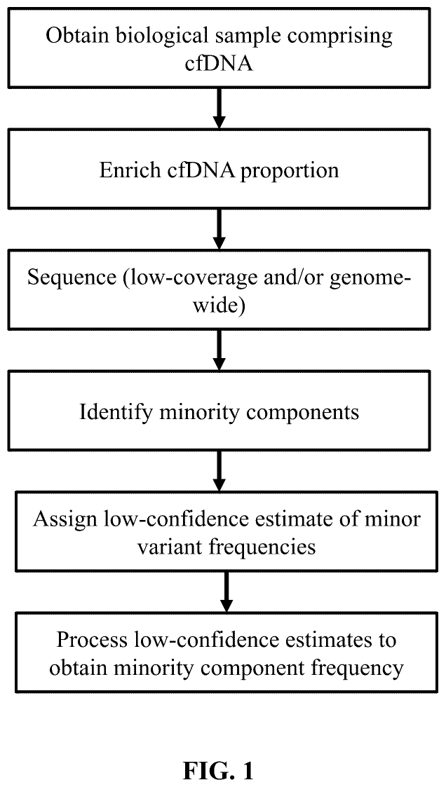

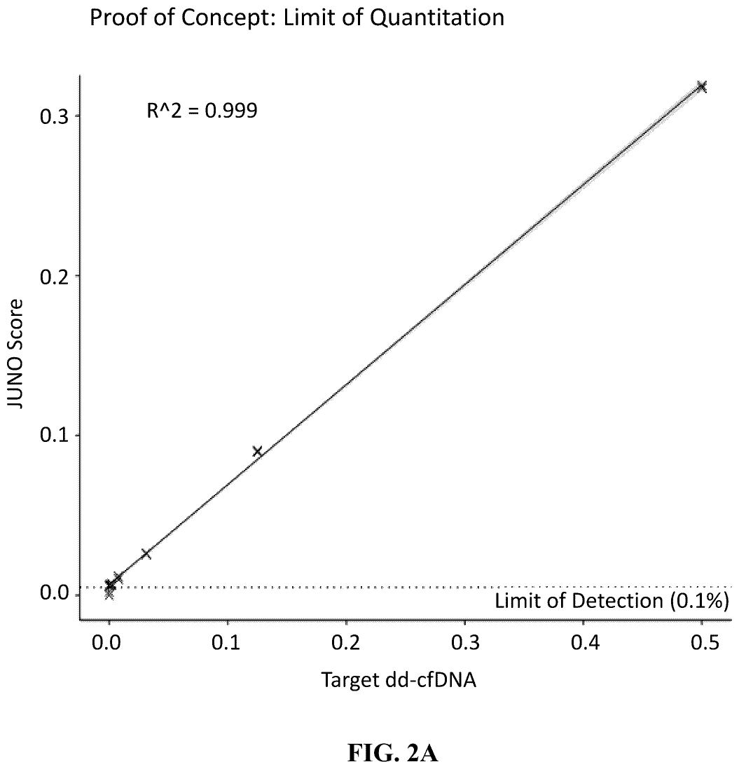

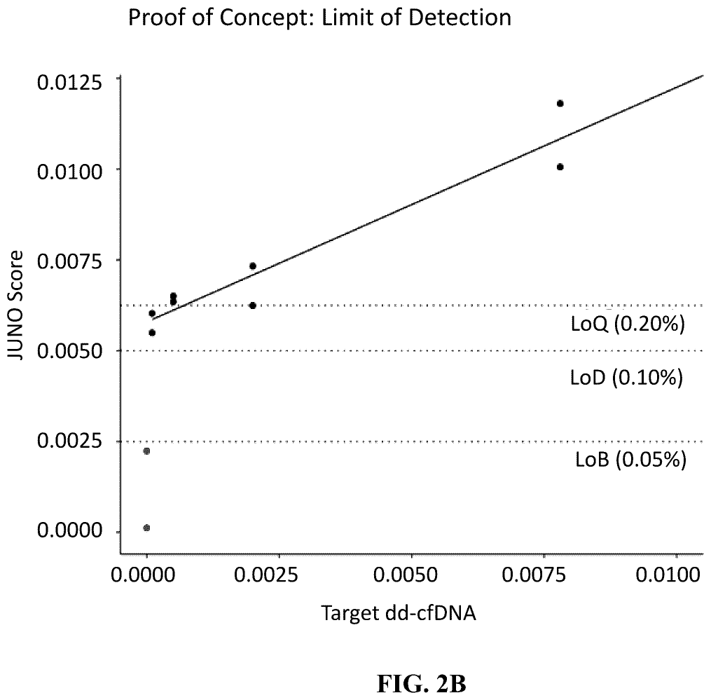

In some aspects, the present disclosure provides a method for analyzing cell free nucleic acids. In some embodiments, the method analyzes cell-free DNA (cfDNA). FIG. 1 shows an illustrative schematic of the method, in accordance with some embodiments. First, a biological sample comprising cfDNA can be obtained from a subject. The biological sample can be optionally enriched such that the proportion of cfDNA in the biological sample is enriched. The sample can be sequenced using a low-coverage and/or genome-wide nucleic acid sequencing method. In some embodiments of methods described herein, the low-coverage nucleic acid sequencing method or low-coverage genome-wide nucleic acid sequencing method comprises sequencing depth sampling the genome being tested at a coverage of between 0.1× and 10×. In some embodiments of methods described herein, the low-coverage nucleic acid sequencing method or low-coverage genome-wide nucleic acid sequencing method comprises sequencing depth sampling the genome being tested at a coverage of about 0.1×, 0.2×, 0.3×, 0.4×, 0.5×, 0.6×, 0.7×, 0.8×, 0.9×, 1×, 1.1×, 1.2×, 1.3×, 1.4×, 1.5×, 1.6×, 1.7×, 1.8×, 1.9×, 2×, 2.25×, 2.5×, 2.75×, 3×, 3.5×, 4×, 4.5×, 5×, 5.5×, 6×, 6.5×, 7×, 7.5×, 8×, 8.5×, 9×, 9.5, or 10×. In some embodiments of methods described herein, the low-coverage nucleic acid sequencing method or low-coverage genome-wide nucleic acid sequencing method comprises sequencing depth sampling the genome being tested at a coverage of between about 0.1× to 2×. In some embodiments of methods described herein, the low-coverage nucleic acid sequencing method or low-coverage genome-wide nucleic acid sequencing method comprises sequencing depth sampling the genome being tested at a coverage of at least about 0.1×, but not greater than about 2×. In some embodiments of methods described herein, the low-coverage nucleic acid sequencing method or low-coverage genome-wide nucleic acid sequencing method comprises sequencing depth sampling the genome being tested at a coverage of less than about 1×, but at least about 0.1×. In some embodiments of methods described herein, the low-coverage nucleic acid sequencing method or low-coverage genome-wide nucleic acid sequencing method comprises sequencing depth sampling the genome being tested at a coverage about IX. The sequencing can generate a dataset from which minority components present in the sequenced cfDNA can be identified. A designation can be assigned that represents a low-confidence estimate of minor variant frequency for individual identified minority components present in the sequenced cfDNA. A plurality of low-confidence estimates of minor variant frequencies can be averaged, e.g., across a plurality of sequenced genomic loci to produce an estimation of minority component frequency in the cfDNA enriched biological sample. The estimation of minority component frequency in the cfDNA enriched biological sample can be a quantitative detection of a minority component present in the cfDNA of the biological sample. The individual identified minority components in cfDNA can comprise alternate heterozygous alleles and/or alternate homozygous alleles when compared to the germline alleles of genomic DNA from the subject. In some embodiments, alternate homozygous alleles from a plurality of loci present in a minority component of cfDNA from a biological sample are identified and distinguished from homozygous germline alleles with a different genotype from a plurality of loci in genomic DNA from the subject. In some embodiments, heterozygous alleles from a plurality of loci present in a minority component of cfDNA from a biological sample are identified and distinguished from homozygous germline alleles with a different genotype from a plurality of loci in genomic DNA from the subject. In some embodiments, alternate heterozygous alleles from a plurality of loci present in a minority component of cfDNA from a biological sample are identified and distinguished from heterozygous germline alleles with a different genotype from a plurality of loci in genomic DNA from the subject. The individual identified minority components in cfDNA can comprise homozygous alleles when compared to the heterozygous or homozygous alleles of genomic DNA from the subject. The individual identified minority components in cfDNA can comprise alternate homozygous alleles when compared to the alleles of genomic DNA from the subject. The individual identified minority components in cfDNA can comprise alternative alleles to those of the majority component of the cfDNA sample. In some embodiments of methods described herein, the identified alternative alleles may be in either heterozygous or homozygous states within the affected cell or minority constituents when compared with the alleles in the majority component of the cfDNA sample.

In some aspects, the biological sample can be enriched such that the proportion of cfDNA in the biological sample is enriched. In some embodiments, the proportion of cfDNA in the biological sample is enriched compared to an amount of nucleic acid derived from cellular nucleic acids. In some embodiments, the proportion of cfDNA in the biological sample is enriched compared to an amount of nucleic acid derived from cellular DNA. In some embodiments, the proportion of cfDNA in the biological sample is enriched compared to an amount of nucleic acid derived from cellular genomic DNA, mitochondrial DNA, or microbiome DNA, or any combination thereof. In some embodiments, the proportion of cfDNA in the biological sample is enriched by purifying cfDNA within the biological sample. In some embodiments, cfDNA is purified to isolate DNA from other nucleic acids within the biological sample. In some embodiments, cfDNA is purified to enrich the proportion of cfDNA to cellular DNA in the biological sample. In some embodiments, cfDNA is enriched in the biological sample by performing DNA purification to produce a sample with higher DNA purity. In some embodiments, cfDNA is enriched in the biological sample by performing DNA purification to produce a sample with a concentrated subset of cfDNA within a sample with higher DNA purity. In some embodiments, an enriched cfDNA sample, a purified cfDNA sample, or a concentrated cfDNA sample is stored prior to sequencing the cfDNA enriched biological sample. In some embodiments, an enriched cfDNA sample comprises a total concentration of at least 100 ng/mL, 75 ng/mL, 50 ng/mL, 40 ng/ml, 30 ng/ml, 20 ng/ml, 15 ng/ml, 10 ng/ml, 7.5 ng/mL, 5.0 ng/ml, 3.5 ng/ml, 2.5 ng/ml, 2.0 ng/ml, 1.5 ng/ml, 1.0 ng/ml, 0.75 ng/ml, 0.5 ng/ml, 0.25 ng/mL, or 0.1 ng/ml of DNA per volume of the biological sample when measured by liquid quantification. In some embodiments, the biological sample is combined with an agent that selectively binds to nucleic acid or a subset of nucleic acid. In some embodiments, the agent that selectively binds to nucleic acid comprises a magnetic beads, silica, carbide, silica carbide, chitosan, a polymer, or a charged material.

In some embodiments, the biological sample to be enriched, such that the proportion of cfDNA in the biological sample, is enriched is obtained from serum, plasma, blood, saliva, urine, mucus, tears, sweat, semen, breast milk, lymphatic fluid, cerebrospinal fluid, or amniotic fluid of the subject. In some embodiments, in which blood is obtained from the subject, peripheral venous blood is collected in an EDTA-containing blood collection tube. In some embodiments, in which blood is obtained from the subject, peripheral venous blood is collected using a blood collection tube that does not contain EDTA or other additives. In some embodiments, in which blood is obtained from the subject, peripheral venous blood is collected using a plasma separation device. In some embodiments, the plasma separation device does not use EDTA or another chelating agent to separate plasma from the blood sample. In some embodiments, in which blood is obtained from the subject, capillary blood is collected using a sample collection and plasma separation card. Enriched cfDNA samples can be produced from any of the sources of sample listed above.

For peripheral blood samples collected via an EDTA-containing blood collection tube, the blood sample can then be centrifuged at 1600×g for 10 minutes at 4° C. in order to separate plasma from peripheral blood cells in the sample. The plasma portion can then be transferred to a new sterile tube and centrifuged at 16,000×g for 10 minutes at 4° C. to pellet any remaining cells in the biological sample. The plasma portion is again transferred to a new sterile tube. cfDNA can be extracted from the purified plasma using a QIAamp DSP DNA Blood Mini Kit (QIAGEN®) following the manufacturer's protocol. Next, the cfDNA enriched biological sample can be end-repaired, adaptor-ligated, and PCR amplified to construct a library for subsequent low-coverage, genome-wide sequencing by using the Ion Xpress™ Plus Fragment Library Kit (Thermo Fisher Scientific). In some embodiments, cfDNA enrichment is performed following end-repairing and before adaptor ligation during construction of the library. Magnetic beads with an average particle size of 1 μm can be used for the purpose of size-selecting the end-repaired DNA fragments. In some embodiments, enriching a proportion of cfDNA within the biological sample comprises selecting cfDNA of a biological sample based on fragment size. In some embodiments, selecting cfDNA comprises segregating cfDNA fragments in the biological sample that are about or less than a certain nucleotide length to obtain a fragment-length enriched population of cfDNA fragments. In some embodiments, the fragment-length enriched population of cfDNA fragments comprises DNA fragments less than about 5000 base-pairs (bp), 2000 bp, 1000 bp, 750 bp, 500 bp, 400 bp, 350 bp, 325 bp, 310 bp, 300 bp, 280 bp, 275 bp, 250 bp, 235 bp, 220 bp, 200 bp, 180 bp, 175 bp, 170 bp, 167 bp, 160 bp, 150 bp, 140 bp, 135 bp, 130 bp, 120 bp, 100 bp, 80 bp, 70 bp, 60 bp, or 50 bp in length. In some embodiments, the fragment-length enriched population of cfDNA fragments comprises DNA fragments greater than about 350 bp, 325 bp, 310 bp, 300 bp, 280 bp, 275 bp, 250 bp, 235 bp, 220 bp, 200 bp, 180 bp, 175 bp, 170 bp, 167 bp, 160 bp, 150 bp, 140 bp, 135 bp, 130 bp, 120 bp, 100 bp, 80 bp, 70 bp, 60 bp, or 50 bp in length. In some embodiments, the fragment-length enriched population of cfDNA fragments comprises DNA fragments in between about 50-350 bp in length. In some embodiments, selecting cfDNA of a biological sample based on size comprises fragmenting a cfDNA sample by using sonication, centrifugation, or enzymatic digestion.

The individual identified minority components in cfDNA can comprise any combination of homozygous alleles, alternate homozygous alleles, and alternate heterozygous alleles at a plurality of loci when compared to the alleles at a plurality of loci of genomic DNA from the subject. The homozygous alleles, the alternate homozygous alleles, and/or the alternative heterozygous alleles can be derived from cfDNA from pre-malignant or malignant cells of the subject. The homozygous alleles, the alternate homozygous alleles, and/or the alternative heterozygous alleles can be derived from circulating tumor DNA (ctDNA). In some embodiments, ctDNA is a component of cfDNA from a biological sample which is shed by malignant tumors into the bloodstream and into other bodily fluids. In some embodiments, ctDNA comprises at least 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.25%, 0.30%, 0.35%, 0.40%, 0.45%, 0.5%, 0.6%, 0.7%, 0.8%, 0.9%, 1.0%, 1.1%, 1.2%, 1.3%, 1.4%, 1.5%, 1.75%, 2.0%, 2.5%, 2.75%, 3.0%, 3.5%, 4.0%, 4.5%, 5%, 6%, 7%, 8%, 9%, or 10% of cfDNA in a biological sample derived from a subject. The homozygous alleles, the alternate homozygous alleles, and/or the alternative heterozygous alleles can be derived from cfDNA from one or more infectious agents residing within the subject. The homozygous alleles, the alternate homozygous alleles, and/or the alternative heterozygous alleles can be derived from a donor subject. The donor subject can be an embryo. The donor subject can be a fetus. The donor subject can be a child or adolescent. The donor subject can be an adult. The donor subject can provide a tissue or an organ transplant into the subject which serves as a host. The donor subject can be Human Leukocyte Antigen (HLA)-matched to the host. The donor subject can be non-consanguineous to the host. The donor subject can be consanguineous with the host. A consanguineous donor subject can have a coefficient of relationship indicating a percentage of shared DNA with the host of about 50%, 37.5%, 25%, 12.5%, 9.38%, 6.25%, 3.13%, 0.78%, or 0.20%. In some embodiments, an estimate of a percentage of shared DNA between the consanguineous donor subject and the host due to the coefficient of relationship is accounted for in the power of statistical calculation for a method of estimating a minority component frequency in cfDNA within a biological sample. The detected minority component present in the cfDNA of the biological sample can comprise less than 25%, 20%, 15%, 12.5%, 10%, 9%, 8%, 7%, 6%, 5%, 4%, 3%, 2%, 1.5%, 1.4%, 1.3%, 1.25%, 1.2%, 1.15%, 1.1%, 1.05%, 1.0%, 0.95%, 0.9%, 0.8%, 0.7%, 0.6%, 0.5%, 0.45%, 0.4%, 0.35%, 0.3%, 0.25%, 0.2%, 0.15%, 0.1%, 0.09%, 0.08%, 0.07%, 0.06%, or 0.05% of the total cfDNA present in the biological sample. The detected minority component present in the cfDNA of the biological sample can comprise at least about 25%, 20%, 15%, 12.5%, 10%, 9%, 8%, 7%, 6%, 5%, 4%, 3%, 2%, 1.5%, 1.4%, 1.3%, 1.25%, 1.2%, 1.15%, 1.1%, 1.05%, 1.0%, 0.95%, 0.9%, 0.8%, 0.7%, 0.6%, 0.5%, 0.45%, 0.4%, 0.35%, 0.3%, 0.25%, 0.2%, 0.15%, 0.1%, 0.09%, 0.08%, 0.07%, 0.06%, or 0.05% of the total cfDNA present in the biological sample. The detected minority component present in the cfDNA of the biological sample can comprise about 25%, 20%, 15%, 12.5%, 10%, 9%, 8%, 7%, 6%, 5%, 4%, 3%, 2%, 1.5%, 1.4%, 1.3%, 1.25%, 1.2%, 1.15%, 1.1%, 1.05%, 1.0%, 0.95%, 0.9%, 0.8%, 0.7%, 0.6%, 0.5%, 0.45%, 0.4%, 0.35%, 0.3%, 0.25%, 0.2%, 0.15%, 0.1%, 0.09%, 0.08%, 0.07%, 0.06%, or 0.05% of the total cfDNA present in the biological sample. The detected minority component present in the cfDNA of the biological sample can comprise a range of about 0.05% to 0.10%, 0.05% to 0.20%, 0.05% to 0.50%, 0.05% to 1.0%, 0.05% to 2.0%, 0.05% to 5%, 0.05% to 10%, 0.05% to 25%, 0.10% to 0.20%, 0.10% to 0.50%, 0.10% to 1.0%, 0.10% to 2.0%, 0.10% to 5%, 0.10% to 10%, 0.10% to 25%, 0.15% to 0.30%, 0.15% to 0.50%, 0.15% to 0.75%, 0.15% to 1.0%, 0.15% to 2.0%, 0.15% to 5%, 0.15% to 10%, 0.15% to 25%, 0.20% to 0.40%, 0.20% to 0.60%, 0.20% to 1.0%, 0.20% to 2.0%, 0.20% to 5%, 0.20% to 10%, 0.20% to 25%, 0.25% to 0.50%, 0.25% to 0.75%, 0.25% to 1.0%, 0.25% to 2.5%, 0.25% to 5%, 0.25% to 10%, 0.25% to 25%, 0.35% to 0.60%, 0.35% to 0.75%, 0.35% to 1.0%, 0.35% to 2.0%, 0.35% to 5%, 0.35% to 10%, 0.35% to 25%, 0.50% to 1.0%, 0.50% to 2.0%, 0.50% to 3.0%, 0.50% to 5%, 0.50% to 7.5%, 0.50% to 10%, 0.50% to 25%, 0.75% to 1.5%, 0.75% to 3.0%, 0.75% to 5%, 0.75% to 10%, 0.75% to 25%, 1.0% to 2.0%, 1.0% to 3.5%, 1.0% to 5%, 1.0% to 7.5%, 1.0% to 10%, 1.0% to 15%, 1.0% to 20%, 1.0% to 25%, 1.5% to 3.0%, 1.5% to 5%, 1.5% to 7.5%, 1.5% to 10%, 1.5% to 15%, 1.5% to 20%, 1.5% to 25%, 2.0% to 5.0%, 2.0% to 7.5%, 2.0% to 10%, 2.0% to 15%, 2.0% to 20%, 2.0% to 25%, 3.5% to 7%, 3.5% to 15%, 3.5% to 20%, or 3.5% to 25% of the total cfDNA present in the biological sample. In some embodiments, the detected minority component present in the cfDNA of the biological sample comprises less than 25%, 20%, 15%, 12.5%, 10%, 9%, 8%, 7%, 6%, 5%, 4%, 3%, 2%, 1.5%, 1.4%, 1.3%, 1.25%, 1.2%, 1.15%, 1.1%, 1.05%, 1.0%, 0.95%, 0.9%, 0.8%, 0.7%, 0.6%, 0.5%, 0.45%, 0.4%, 0.35%, 0.3%, 0.25%, 0.2%, 0.15%, 0.1%, 0.09%, 0.08%, 0.07%, 0.06%, or 0.05% of the total cfDNA present in the biological sample.

In some cases, the method can comprise absolute quantification of the minority component. The identified minority component alternate homozygous alleles or alternative heterozygous alleles can comprise less than about 2000, 1500, 1250, 1000, 900, 800, 750, 600, 500, 400, 300, 200, 150, 125, 110, 100, 90, 85, 80, 75, 70, 65, 60, 55, 50, 45, 40, 35, 34, 33, 32, 31, 30, 29, 28, 27, 26, 25, 24, 23, 22, 21, 20, 19, 18, 17, 16, 15, 14, 13, 12, 11, or 10 loci. The identified minority component alternate homozygous alleles or alternative heterozygous alleles can comprise greater than about 2000, 1500, 1250, 1000, 900, 800, 750, 600, 500, 400, 300, 200, 150, 125, 110, 100, 90, 85, 80, 75, 70, 65, 60, 55, 50, 45, 40, 35, 34, 33, 32, 31, 30, 29, 28, 27, 26, 25, 24, 23, 22, 21, 20, 19, 18, 17, 16, 15, 14, 13, 12, 11, or 10 loci. The identified minority component alternate homozygous alleles or alternative heterozygous alleles can comprise about 2000, 1500, 1250, 1000, 900, 800, 750, 600, 500, 400, 300, 200, 150, 125, 110, 100, 90, 85, 80, 75, 70, 65, 60, 55, 50, 45, 40, 35, 34, 33, 32, 31, 30, 29, 28, 27, 26, 25, 24, 23, 22, 21, 20, 19, 18, 17, 16, 15, 14, 13, 12, 11, or 10 loci. The identified minority component alternate homozygous alleles can comprise less than about 1000, 750, 500, 400, 300, 200, 150, 125, 110, 100, 90, 80, 75, 70, 65, 60, 55, 50, 45, 40, 35, 30, 25, or 20 loci. The identified minority component alternate homozygous alleles can comprise less than about 1000, 750, 500, 400, 300, 200, 150, 125, 110, 100, 90, 80, 75, 70, 65, 60, 55, 50, 45, 40, 35, 30, 25, or 20 SNP loci. The identified minority component alternate homozygous alleles can comprise greater than about 1000, 750, 500, 400, 300, 200, 150, 125, 110, 100, 90, 80, 75, 70, 65, 60, 55, 50, 45, 40, 35, 30, 25, or 20 loci. The identified minority component alternate homozygous alleles can comprise about 1000, 750, 500, 400, 300, 200, 150, 125, 110, 100, 90, 80, 75, 70, 65, 60, 55, 50, 45, 40, 35, 30, 25, or 20 loci. In some embodiments, the loci comprise SNP loci. In some embodiments, the loci comprise small insertions or deletions (INDELs). In some embodiments, the loci comprise variable number of tandem repeats (VNTRs). In some embodiments, the loci comprise simple sequence repeats (SSRs). In some embodiments, the loci comprise simple tandem repeats (STRs). In some embodiments, the loci comprise SNPs, INDELs, VNTRs, SSRs, or STRs, or any combination thereof. In some embodiments, the loci are SNP loci. In some embodiments, the loci consist of SNP loci.

In some embodiments, the method analyzes cell-free RNA (cfRNA). In some embodiments, the method analyzes cell-free mRNA, cell-free tRNA, cell-free rRNA, or any combination thereof. In some embodiments, the cfRNA has been released from cancerous and non-cancerous cells. In some embodiments, the method analyzes cell-free RNA alternative splicing configurations. In some embodiments, the cfRNA is derived from one or more non-transformed tissues. In some embodiments, the one or more non-transformed tissues comprises stroma for an organ, gland, or tissue. In some embodiments, the one or more non-transformed tissues comprise a hematopoietic tissue. In some embodiments, the cfRNA is obtained from a biological sample from the host subject. In some embodiments, the biological sample is from serum, plasma, blood, saliva, urine, mucus, tears, sweat, semen, breast milk, lymphatic fluid, cerebrospinal fluid, or amniotic fluid of the host subject. In some embodiments, the cfRNA obtained from the biological sample is enriched. In some embodiments, the cfRNA obtained from the biological sample is purified. In some embodiments, the cfRNA obtained from the biological sample is reversed transcribed into cDNA for further analysis using low-coverage, genome-wide sequencing, exome sequencing, or targeted sequencing. In some embodiments, alternative heterozygous and/or alternative homozygous alleles are detected and analyzed in a minority component of cfRNA compared to the majority component of cfRNA in the sample. In some embodiments, alternative heterozygous and/or alternative homozygous alleles are detected and analyzed in a minority component of cell-free mRNA compared to the majority component of cell-free mRNA in the sample. In some embodiments, the alternative heterozygous and/or alternative homozygous alleles comprise SNPs. In some embodiments, the alternative heterozygous and/or alternative homozygous alleles comprise INDELs. In some embodiments, alternative alleles comprising alternatively spliced mRNA transcripts are detected and analyzed in a minority component of cell-free mRNA compared to the majority component of cell-free mRNA in the sample.

Fragment patterning can be analyzed in the cfDNA enriched biological sample. In some embodiments, fragment patterning can be analyzed following PCR amplification. In some embodiments, PCR amplified fragments can be analyzed by electrophoresis (e.g., gel or capillary electrophoresis). In some embodiments, fragment patterning can be analyzed by assessing restriction fragment length polymorphisms (RFLP). In some embodiments, RFLP analysis uses an RFLP probe comprising a labeled DNA sequence that can hybridize with one or more fragments of DNA sample following digestion with one or more restriction endonucleases following separation by electrophoresis. In some embodiments, fragment patterning can be analyzed by amplified fragment length polymorphism analysis. In some embodiments, fragment patterning can be analyzed by single-strand conformation polymorphism analysis. In some embodiments, fragment patterning can be analyzed by next generation sequencing.

Missing SNP genotypes in the sequenced cfDNA enriched biological sample can be imputed. Genotype imputation is the process of inferring unobserved genotypes in a sample or in a plurality of samples. Regional linkage disequilibrium ratios can be calculated and evaluated between variant alleles detected to enhance the calculated estimation of minority component frequency in the cfDNA enriched biological sample. In some embodiments, a haplotyping program is used to impute missing genotypes during haplotype estimation. In some embodiments, a genotype imputation tool such as PLINK, TUNA, WHAP, or BEAGLE is used to impute genotypes by focusing the analysis on a relatively small number of nearby markers when imputing a missing genotype. In some embodiments, a genotype imputation tool such as IMPUTE, MACH, or fastPHASE/BIMBAM is used to impute genotypes by taking into account all observed genotypes when imputing each missing genotype.

The method can be tuned to individual subjects, based on longitudinal sampling of biological samples obtained from the subject to screen for change in minor cfDNA components over time. In some embodiments, longitudinal sampling involves tuning of the sampling to individual parameters dependent on the subject being assessed. In some embodiments, longitudinal sampling is compressed for a subject exhibiting a severe disease or for a subject exhibiting a rapid worsening of a condition. In some embodiments, the compression of longitudinal sampling comprises reducing a total number of samples collected for the longitudinal analysis and/or increased the frequency of sampling. In some embodiments, longitudinal sampling comprises collecting at least 2, 3, 4, 5, 6, 7, 8, 9, 10, 11, 12, 13, 14, 15, 16, 17, 18, 19, 20, 25, 30, 35, 40, 45, or 50 biological samples from the subject at different points in time. In some embodiments, the biological samples are collected approximately every 1, 2, 3, 4, 5, 6, 7, 8, 9, 10, 11, 12, 13, 14, 15, 16, 17, 18, 19, 20, 21, 25, 28, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, or 365 days. The low-coverage, genome-wide nucleic acid sequencing can be unbiased sequencing. The low-coverage, genome-wide nucleic acid sequencing can be hypothesis-free, e.g., untargeted sequencing.

Genomic DNA derived from a subject can be analyzed and used as a reference sequence. The genomic DNA derived from the subject can be used to distinguish germline genotypes present in the subject from somatic genotypes as minority components in the cfDNA enriched biological sample. The minority components may be somatic variants arising due to cellular disease states (e.g., cancer or immune-related disorders). The minority components may be DNA alternative to germline DNA of the subject that arises due to circumstances specific to the subject (e.g., pregnancy or organ transplantation). The somatic genotypes can be identified through high confidence genotyping. The high confidence genotyping can comprise sequencing of at least 2×, 3×, 4×, 5×, 6×, 7×, 8×, 9×, 10×, 12×, 15×, or 20× genomic coverage. In some embodiments, somatic genotypes can be identified through targeted deep sequencing, next generation sequencing methods, digital PCR, or allele-specific PCR.

For methods using next generation sequencing, individual base call (BCL) files are generated. Following completion of next generation sequencing, BCL files are converted into raw sequence data by generating FASTQ files and FASTQ files are assessed for quality control. Next, FASTQ are trimmed by using a program such as Skewer, Cutadapt, or Trimmomatic to remove low quality bases and adapter sequences from sequencing reads. The trimmed, raw sequence data can be aligned to a reference sequence (e.g., aligned with a human genome reference sequence represented in UCSC Genome Browser, Ensembl genome browser, or IGV (Broad Institute)). The raw sequence can be generated with low-coverage sequencing. The raw sequence can be generated with whole genome sequencing. The raw sequence can be generated with transcriptome sequencing. The raw sequence can be generated with low-coverage whole-genome sequencing (lcWGS). The low-coverage sequencing can be performed without amplification, e.g., without PCR. Duplicate reads of sequenced fragments can be marked. The low-coverage sequencing can comprise less than about 5×, 4.5×, 4×, 3.5×, 3×, 2.5×, 2.25×, 2×, 1.75×, 1.6×, 1.5×, 1.4×, 1.3×, 1.2×, 1.1×, 1×, 0.9×, 0.8×, 0.7×, 0.6×, 0.5×, 0.4×, 0.35×, 0.3×, 0.25×, 0.2×, 0.15×, 0.125×, 0.1×, 0.09×, 0.08×, 0.075×, 0.065×, 0.05×, 0.035×, 0.025×, 0.02×, 0.015×, 0.01×, or 0.005× sequencing coverage of the genome of the subject. The low-coverage sequencing can comprise greater than about 5×, 4.5×, 4×, 3.5×, 3×, 2.5×, 2.25×, 2×, 1.75×, 1.6×, 1.5×, 1.4×, 1.3×, 1.2×, 1.1×.0.9×, 0.8×, 0.7×, 0.6×, 0.5×, 0.4×, 0.35×, 0.3×, 0.25×, 0.2×, 0.15×, 0.125×, 0.1×, 0.09×, 0.08×, 0.075×, 0.065×, 0.05×, 0.035×, 0.025×, 0.02×, 0.015×, 0.01×, or 0.005× sequencing coverage of the genome of the subject. In some embodiments, the sequencing comprises real-time analysis.

Generated genomic datasets (e.g., BAM files) can be pre-processed. The pre-processing can comprise base quality score recalibration (BQSR). Then, sequences can be locally aligned to produce analysis-ready BAM files. The sites sequenced with low-coverage, genome-wide nucleic acid sequencing can be agnostic to pre-defined genomic loci. The sites sequenced with low-coverage, genome-wide nucleic acid sequencing can be targeted to pre-defined genomic loci. Variant calling can be performed on the analysis-ready BAM to identify positions (e.g., SNPs or INDELs) that differ from a reference. The variants detected comprise single-nucleotide polymorphisms (SNPs), small insertions or deletions (INDELs), variable number of tandem repeats (VNTR), simple sequence repeats (SSR), or simple tandem repeats (STR), or any combination thereof. The variants can comprise SNPs. The number of genomic loci assayed by the low-coverage, genome-wide nucleic acid sequencing to detect potential variants can be at least 5000, 10000, 20000, 35000, 50000, 75000, 100000, 150000, 200000, 250000, 300000, 400000, 500000, 600000, 750000, 875000, 1000000, 2000000, 3000000, 4000000, 5000000, 7500000, or 10000000 genomic loci. The number of genomic loci assayed by the low-coverage, genome-wide nucleic acid sequencing to detect potential variants can be at most 5000, 10000, 20000, 35000, 50000, 75000, 100000, 150000, 200000, 250000, 300000, 400000, 500000, 600000, 750000, 875000, 1000000, 2000000, 3000000, 4000000, 5000000, 7500000, or 10000000 genomic loci. Low coverage differences in variants identified that differ from a reference allows the detection, identification, and quantitation of low level minority components in the cfDNA or cfRNA pool derived from the sample.

The designation can be a classifier for the individual identified minority components. The classifier can be binary classifier. The classifier can be a non-binary classifier. The designation can be a likelihood for the individual identified minority components. The designation can be a machine learning algorithm. A machine learning algorithm can be a neural network. A machine learning algorithm can be a random forest algorithm. The individual identified minority components can be preprocessed, for example, normalized before classifying or determining a likelihood. The classifier can distinguish a sequenced genomic locus that originates from the minority component. The classifier can distinguish a sequenced genomic locus that originates from the non-minority component. The classifier can distinguish a plurality of sequenced genomic loci from sequenced genomic loci not identified as having a minor variant in the sequenced cfDNA enriched biological sample.

The biological sample can be obtained by non-invasive, minimally-invasive, or an invasive procedure. The biological sample can be obtained using a kit disclosed herein. The biological sample can be obtained using a blood draw, phlebotomy, throat swab, buccal swab, bronchial lavage, urine collection, skin or epidermal scraping, feces collection, menses collection, semen collection, blood draw, venipuncture, biopsy, alveolar or pulmonary lavage, needle aspiration, fingerstick, dried blood spot, capillary-based collection or any combination thereof. The biological sample can be from serum, plasma, blood, saliva, urine, mucus, tears, sweat, semen, breast milk, lymphatic fluid, cerebrospinal fluid, or amniotic fluid of the subject. The biological sample can be from plasma or blood of the subject. The volume of the biological sample obtained from the subject can be less than about 1000 μL, 900 μL, 800 μL, 700 μL, 600 μL, 500 μL, 400 μL, 300 μL, 200 μL, 150 μL, 125 μL, 100 μL, 80 μL, 75 μL, 70 μL, 60 μL, 55 μL, 50 μL, 45 μL, 40 μL, 35 μL, 30 μL, 25 μL, 20 μL, 17.5 μL, 15 μL, 12.5 μL, 10 μL, 9 μL, 8 μL, 7 μL, 6 μL, 5 μL, 4 μL, 3 μL, 2.5 μL, 2 μL, 1.5 μL, 1 μL, or 0.5 μL. In some embodiments, in which blood is obtained from the subject, the volume of blood obtained from the biological sample derived from a subject for use in the method for analyzing cfDNA is less than about 10 mL, 8 mL, 7.1 mL, 6.1 mL, 4.6 mL, 2.1 mL, 1 mL, 0.75 mL, 500 μL, 400 μL, 350 μL, 300 μL, 275 μL, 250 μL, 225 μL, 200 μL, 175 μL, 150 μL, 125 μL, 100 μL, 80 μL, 75 μL, 70 μL, 65 μL, 60 μL, 55 μL, 50 μL, 45 μL, 40 μL, 35 μL, 30 μL, 25 μL, 20 μL, 17.5 μL, 15 μL, 12.5 μL, 10 μL, 9 μL, 8 μL, 7 μL, 6 μL, 5 μL, 4 μL, 3 μL, 2.5 μL, 2 μL, 1.5 μL, 1 μL, or 0.5 μL. The volume of the biological sample obtained from the subject can be greater than about 200 μL, 150 μL, 125 μL, 100 μL, 80 μL, 75 μL, 70 μL, 60 μL, 55 μL, 50 μL, 45 μL, 40 μL, 35 μL, 30 μL, 25 μL, 20 μL, 17.5 μL, 15 μL, 12.5 μL, 10 μL, 9 μL, 8 μL, 7 μL, 6 μL, 5 μL, 4 μL, 3 μL, 2.5 μL, 2 μL, 1.5 μL, 1 μL, or 0.5 μL.

The method can further comprise imputing missing genotypes and may improve statistical power by permitting inference of additional genotypes while still using low-coverage genome-wide sequencing approaches. The method can comprise utilizing fragment patterning to improve accuracy and/or the limit of detection. The method can comprising utilizing longitudinal sampling to improve accuracy of minority component concentration and to identify changes in disease state or condition in a subject over the time period of longitudinal sampling. The biological underpinnings of cfDNA generation from tissues are critical to understanding the utility of cfDNA approaches in clinical practice. While SNP profiling may clearly identify origins of minority cfDNA components, the fragment sizes, fragment end-position biases, etc., may additionally inform the likely origin and better refine estimates of minor component frequency in cfDNA. The method can comprise utilizing epigenetic markers to improve accuracy and/or the limit of detection. Epigenetic markers can inform tissue of origin in cfDNA for oncology applications and prenatal screening. Additional information concerning epigenetic marks (e.g., methylation) may be used to refine the likelihood estimates of minor component frequencies in cfDNA. The method can comprise learned patient-level fingerprints to improve accuracy and/or the limit of detection. Data from longitudinal sampling of patients to screen for change in minor cfDNA components over time can be used to “learn” an individual level fingerprint of both SNP distributions and the features described above to further refine estimation of the frequency of the minor components of cfDNA.

### Transplant Monitoring

The terms “transplantation” or a “transplant” can refer to the transfer of tissues, cells, or a solid organ from a donor individual into a recipient individual. A donor and recipient may or may not be from the same species. For example, a human recipient may receive a solid organ from a non-human animal in some embodiments. An “allograft” further indicates a transfer of tissues, cells, or a solid organ between different individuals of the same species. In contrast, if the donor and recipient are the same individual, the graft can be referred to as an “autograft.” The estimation of minority component frequency in the cfDNA enriched biological sample can be used for transplantation monitoring.

A transplant can comprise an organ or tissue transplant. A transplant can comprise adrenal gland, appendix, bladder, brain, ear, esophagus, eye, gall bladder, heart, kidney, large intestine, liver, lung, mouth, muscle, nose, pancreas, parathyroid gland, pineal gland, pituitary gland, skin, small intestine, spleen, stomach, thymus, thyroid gland, trachea, uterus, vermiform appendix, cornea, skin, heart valve, artery, or vein. In some cases, the organ is a gland organ. For example, the organ may be an organ of the digestive or endocrine system; in some cases, the organ can be both an endocrine gland and a digestive organ. The organ may be derived from endoderm, ectoderm, primitive endoderm, or mesoderm. The transplant can be a vascularized composite allograft transplant.

The organ, tissue or cell transplant can be an intact organ, a fragment of an intact organ, a disrupted organ, or a cell from any of the organs disclosed herein. Donor cells may be derived from any of the donor organs disclosed herein (e.g., pancreatic cell, hepatic cell, glioma, etc.). The transplanted tissue may also comprise stem cells (e.g., multipotent stem cells, pluripotent stem cells, neuronal stem cells, heart stem cells, induced pluripotent stem cells, embryonic stem cells, cells derived from cord blood, etc.). In some cases, the transplant organ, tissues or cells may comprise cholecystocytes, cardiomyocytes, valve cells, glomerulus cells (e.g., parietal, podocyte), kidney proximal tubule brush border cells, Loop of Henle thin segment cells, thick ascending limb cells, kidney distal tubule cells, kidney collecting ductal cells, or interstitial kidney cells, enterocytes, goblet cells, enterocytes, caveolated tuft cells, enteroendocrine cells, ganglion neurons, parenchymal cells, non-parenchymal cells, hepatocytes, sinusoidal endothelial cells, Kupffer cells, hepatic stellate cells, tendon, cartilage, bone, blood, lymph, myocytes, muscle fibers, pancreatic beta cells, endothelial cells, or exocrine cells. Tissues can include, but are not limited to, connective tissue, epithelial tissue, muscular tissue, nervous tissue, fat tissue, dense fibrous tissue, skeletal muscle, cardiac muscle, or smooth muscle. The muscle tissue may comprise muscle fibers or myocytes. In some cases, the tissue is a bone or tendon (both referred to as musculoskeletal grafts).

The transplant can be a cellular allograft, e.g., a transplant comprising allogeneic cells that originate from a donor. The transplant can comprise cells taken from a donor for administration into a recipient, cells taken from a donor and genetically engineered before administration into a recipient, cells taken from a donor and cultured before administration into a recipient, cells taken from a donor and subjected to a manufacturing process before administration into a recipient, and any combination thereof.

The recipient of the transplant may receive one or more of a variety of allogeneic cells. Allogeneic cells may include, but are not limited to, blood cells, stem cells, cardiomyocytes, neurons, lymphocytes, NK cells, NKT cells, T reg cells, macrophages, dendritic cells, and pancreatic islet cells. In some embodiments, the allogeneic cells are allogeneic blood cells. Allogeneic blood cells may include bematopoietic stem cells (i.e., HSCs), T cells, B cells, and CAR T cells, NK cells, NKT cells, TILs. In some embodiments, the allogeneic cells are allogeneic T cells. In some embodiments, allogeneic cells are administered as bone marrow, cord blood, or purified allogeneic cells. In some embodiments, the allogeneic cells are bone marrow cells. In some embodiments, the allogeneic cells are cord blood cells. In some embodiments, the transplant comprises HSCs. In some embodiments, the HSCs are administered as bone marrow, cord blood, or purified HSCs. In some embodiments, the HSCs are derived from a donor. In some embodiments, the HSCs are administered as a hematopoietic cell transplantation.

A transplant can comprise stem cells. Allogeneic stem cells can be embryonic, tissue-specific, mesenchymal, induced pluripotent, hematopoietic, mesenchymal, skeletal, myogenic, cardiac, neural, epidermal, or intestinal stem cells. In some embodiments, the allogeneic stem cells are hematopoietic stem cells.

A transplant can comprise a cellular graft of autologous cells, e.g., a transplant comprising cells that originate from the recipient. These include, but are not limited to, cells taken from the recipient and genetically engineered before re-administration into the same recipient, cells taken from the recipient and cultured before re-administration into the same recipient, cells taken from the recipient and subjected to a manufacturing process before re-administration into the same recipient, and any combination thereof. For example, immune cells such as lymphocytes, NK cells, or macrophages can be genetically engineered and used to target and kill specific cancer cells. For example, T cells can be modified to produce special structures called chimeric antigen receptors (CARs) on their surfaces that are engineered to target specific cancer antigens; when these CAR T cells are administered into a recipient patient, the CAR receptors can allow the CAR T cells to latch onto their target cancer antigens to kill the cancerous cells while leaving healthy tissues unharmed.

Autologous cells can include blood cells, stem cells, cardiomyocytes, neurons, lymphocytes, NK cells, NKT cells, T reg cells, macrophages, dendritic cells, and pancreatic islet cells, that are genetically engineered and/or subjected to a manufacturing process and/or cultured before re-administration into the same recipient. In some embodiments, the autologous cells are autologous T cells and, following genetic engineering, re-administered as CAR (chimeric antigen receptor) T cells.

The donor organ, tissue, or cells can be derived from a subject who has certain similarities or compatibilities with the recipient subject. For example, the donor organ, tissue, or cells may be derived from a donor subject who is age-matched, ethnicity-matched, gender-matched, blood-type compatible, or HLA-type compatible with the recipient subject. In some circumstances, the donor organ, tissue, or cells may be derived from a donor subject that has one or more mismatches in age, ethnicity, gender, blood-type, or HLA markers with the transplant recipient due to organ availability. The organ may be derived from a living or deceased donor.

A “recipient” can refer to an individual receiving a transplant, allograft, or autograft. A recipient can be a human. A recipient can be monitored using the methods disclosed herein for at least 6 hours, 12 hours, 1 day, 2 days, 3 days, 4 days, 5 days, 10 days, 15 days, 20 days, 25 days, 1 month, 2 months, 3 months, 4 months, 5 months, 7 months, 9 months, 11 months, 1 year, 2 years, 4 years, 5 years, 10 years, 15 years, 20 years. A recipient can be monitored using the methods disclosed herein for at most 6 hours, 12 hours, 1 day, 2 days, 3 days, 4 days, 5 days, 10 days, 15 days, 20 days, 25 days, 1 month, 2 months, 3 months, 4 months, 5 months, 7 months, 9 months, 11 months, 1 year, 2 years, 4 years, 5 years, 10 years, 15 years, 20 years. A recipient can be administered an immunosuppressive drug if transplant rejection is detected. A recipient can receive higher doses of an immunosuppressive drug if transplant rejection is detected. A recipient can receive a different immunosuppressive drug if transplant rejection is detected. If transplant rejection is detected, a surveillance biopsy can be performed for the recipient. If transplant rejection is detected, a high coverage method can be performed to confirm rejection.

Rejection can be an “acute rejection”. An acute rejection can refer to a condition that occurs when transplanted tissue is rejected by the recipient's immune system, which damages or destroys the transplanted tissue unless immunosuppression is achieved. T-cells, B-cells and other immune cells as well as possibly antibodies of the recipient may cause the graft cells to lyse or produce cytokines that recruit other inflammatory cells, eventually causing necrosis of allograft tissue. In some instances, acute rejection can be diagnosed by a biopsy of the transplanted organ. AR can occur in the first three to 12 months after transplantation. AR can also occur for the first five years post-transplant, or whenever a patient's immunosuppression becomes inadequate for any reason for the life of the transplant.

Rejection can be a cellular rejection or an antibody-mediated rejection. In some cases, there may be no evidence of rejection. In some cases, there may be no cellular evidence of rejection. In some cases, there may be no histological evidence of rejection. In some cases, there may be no symptoms of rejection.

### Oncological Detection and Monitoring

The estimation of minority component frequency in the cfDNA enriched biological sample can be used for oncology detection or oncology monitoring. At least two biological samples derived from the subject at different time points can be analyzed to screen for a change in minor cfDNA components over time. Increases the minority component (e.g., the minimal residual disease) can indicate recurrence of cancer. A significant increase in somatic variants over time can be detected, and new somatic variants can be detected and identified in a biological sample collected from the subject after collection of an initial biological sample from the subject. Somatic SNPs can be distinguished from germline SNPs in the subject. An initial biological sample can be obtained from a tumor biopsy. Patterns of DNA methylation in the cfDNA enriched biological sample can be analyzed. Optionally, oncological detection and monitoring can further comprise an analysis of patterns of DNA methylation in the cfDNA enriched biological sample to increase detection accuracy, sensitivity, and/or specificity of ctDNA within the cfDNA enriched biological sample. In some embodiments, the patterns of DNA methylation in the cfDNA enriched biological sample indicate positive tumorigenic transformation, cancer progression, cancer metastasis, or any combination thereof. In some embodiments, detection of global DNA hypomethylation in the cfDNA enriched biological sample indicates positive malignant transformation, cancer progression, cancer metastasis, or any combination thereof. Optionally, oncological detection and monitoring can further comprise an analysis of patterns of DNA strandedness in the cfDNA enriched biological sample. In some embodiments, an increase in a proportion of single-stranded cfDNA in the cfDNA enriched biological sample compared to the amount of double-stranded cfDNA indicates positive malignant transformation, cancer progression, cancer metastasis, or any combination thereof.

By using low-coverage genome-wide sequencing approaches instead of targeted deep sequencing approaches, performance can be improved via assessment of a number of additional features available to genome-wide data collection. In each of these cases, the approach would be to additionally weight an assessment of the frequency of minor components of cfDNA by using non-genotypic data gathered in the course of whole-genome sequencing of cfDNA (e.g., patterns of DNA methylation in cfDNA, detection of global DNA hypomethylation in cfDNA, and analysis of patterns of DNA strandedness in cfDNA).

### Monogenic Disease Screening and Detection

The estimation of minority component frequency in the cfDNA enriched biological sample can be used for monogenic disease screening. The estimation of minority component frequency in the cfDNA enriched biological sample can be used for monogenic disease detection. In some embodiments, the methods described herein may be used for prenatal screening of one or more monogenic diseases. In some embodiments, the methods described herein may be used for prenatal detection of a monogenic disease. In some embodiments, the noninvasive nature of the sample collection techniques used in methods described herein is advantageous for prenatal screening of one or more monogenic diseases or prenatal detection of a monogenic disease. In some embodiments, the methods described herein may be used for noninvasive prenatal diagnosis (NIPD) as an alternative to invasive methods for prenatal diagnosis such as amniocentesis or chorionic villus sampling. In some embodiments, monogenic disease screening and monogenic disease detecting may involving assaying for and identifying one or more potential maternal, paternal, biparental, or de novo mutations. In some embodiments, NIPD may involving collecting and assaying a biological sample from a pregnant subject at approximately 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41 weeks of gestation. In some embodiments, longitudinal sampling is utilized. In some embodiments, the methods described herein may be used to screen for or detect diseases or groups of diseases comprising cystic fibrosis, Huntington's disease, myotonic dystrophy type I, Duchene muscular dystrophy, facioscapulohumeral muscular dystrophy, Gaucher disease, Pompe disease, Friedreich's ataxia, congenital deafness, familial hypercholesterolemia, hemochromatosis, sickle cell disease, Tay-Sachs disease, adrenoleukodystrophy, hemophilia, Adrenal hyperplasia due to 21-hydroxylase deficiency (21-OHD CAH), Aicardi-Goutières syndrome encephalopathy, Alpha-1-antitrypsin (A1AT) deficiency (AATD), Arrhythmogenic right ventricular cardiomyopathy/dysplasia (ARVC, ARVD), Autosomal dominant polycystic kidney disease (ADPKD), Brugada syndrome ventricular fibrillation, Catecholaminergic polymorphic ventricular tachycardia (CPVT), Charcot-Marie-Tooth disease/Hereditary motor and sensory neuropathy, Congenital adrenal hyperplasia (CAH), Congenital sucrase-isomaltase deficiency (CSID), Congenital bilateral absence of vas deferens, Cystinuria-lysinuria syndrome/Cystinuria. Cytomegalic congenital adrenal hypoplasia (AHC) (subtype of congenital adrenal hypoplasia), Dentinogenesis imperfecta (DGI), Dysbetalipoproteinemia/Hyperliproteinemia type 3, Ehlers-Danlos syndrome, Familial adenomatous polyposis (FAP), Gardner syndrome (subtype of familial adenomatous polyposis), Familial cerebral cavernous malformation, Familial hypocalciuric hypercalcemia type 1 (FHH), Familial isolated dilated cardiomyopathy, Familial long QT syndrome (LQTS), including Romano-Ward syndrome, Fragile X syndrome/Martin-bell syndrome, Glucose-6-phosphate dehydrogenase deficiency, GM2 gangliosidosis, Hemolytic anemia due to red cell pyruvate kinase deficiency, Hemophilia A and B, Hemorrhagic telangiectasia/Osler Weder Rendu disease, Hereditary angioedema (HAE)/Angioneurotic edema, Hereditary breast and ovarian cancer syndrome, Hereditary fructose intolerance/Fructosemia, Hereditary xanthinuria/Xanthine stone disease, Hypohidrotic ectodermal dysplasia (HED), Iminoglycinuria, Li-Fraumeni syndrome sarcoma, breast, leukemia, and adrenal gland (SBLA) syndrome, Long chain 3-hydroxyacyl-CoA dehydrogenase deficiency (LCHAD), Lynch syndrome, Marfan syndrome, Maternal phenylketonuria/Phenylketonuric embryopathy, Medium chain acyl-CoA dehydrogenase deficiency (MCADD), Mucolipidosis type III (ML3) alpha/beta, Mucopolysaccharidosis type 4A (MPS4A)/Morquio disease type A, Multiple endocrine neoplasia type 2, Multiple epiphyseal dysplasia (MED), Neurofibromatosis type 1 (NF1)/Von Recklinghausen disease, Oculocutaneous albinism (OCA), Osteogenesis imperfecta/brittle bone disease, Pendred syndrome (PDS)/Deafness with goiter, Phenylketonuria (PKU)/Phenylalanine hydroxylase deficiency (PAH deficiency), Proximal spinal muscular atrophy (SMA), Retinitis Pigmentosa (RP), Recessive X-linked ichthyosis (XLI), Retinoblastoma, Rett syndrome, Sotos syndrome/cerebral gigantism, Stargardt disease/Fundus flavimaculatus, Stickler syndrome/hereditary progressive arthroophthalmopathy, Supravalvular aortic stenosis (SVAS), β-Thalassemia, Tibial muscular dystrophy/Upp myopathy, Tuberous sclerosis complex/Bourneville syndrome, Von-Hippel Lindau disease. Von Willebrand disease, X-linked adrenoleukodystrophy (ALD), and X-linked retinoschisis (XLRS).

### Kits

In some aspects, the present disclosure provide kits for use in the quantitative identification of minority components that are present in cell-free nucleic acids. A kit can comprise one or more biological sample collection devices. Samples may be obtained longitudinally. The sample can be obtained about every 1, 2, 3, 4, 5, 6, 7, 8, 9, 10, 11, or 12 hours. The sample can be obtained about every 1, 2, 3, 4, 5, 6, or 7 days. The sample can be obtained about every 1, 2, 3, 4 or 5 weeks. The sample can be obtained about every 1, 2, 3, 4, 5, 6, 7, 8, 9, 10, 11, or 12 months.

In some aspects disclosed herein are kits for obtaining genetic information from a biological sample. A kit can allow a user to collect and test a biological sample at a location of choice to detect the presence and/or quantity of a minority component in the sample. In some instances kits comprise a sample purifier that removes at least one component (e.g., cell, cell fragment, protein) from a biological sample of a subject. A kit can comprise a nucleic acid sequencer for sequencing cell-free nucleic acids in the biological sample. A kit can comprise a nucleic acid sequence output for relaying sequence information to a user.

A kit can integrate multiple functions, e.g., purification, amplification, and detection of the target analyte (e.g., cfDNA), and combinations thereof. In some instances, the multiple functions are carried out within a single assay assembly unit or a single device. In some instances, all of the functions occur outside of the single unit or device. In some instances, at least one of the functions occurs outside of the single unit or device. In some instances, only one of the functions occurs outside of the single unit or device. In some instances, the sample purifier, oligonucleotide, and detection reagent or component are housed in a single device. A kit can comprise a display, a connection to a display, or a communication to a display for relaying information about the biological sample to one or more people.

A kit can comprise an additional component such as a sample transportation compartment, a sample storage compartment, a sample and/or reagent receptacle, a temperature indicator, an electronic port, a communication connection, a communication device, a sample collection device, a housing unit, or any combination thereof. In some instances, the additional component is integrated with the device. In some instances, the additional component is not integrated with the device. In some instances, the additional component is housed with the sample purifier, detection reagent, or component in a single device. In some instances, the additional component is not housed within the single device.

Kits can comprise components to obtain a sample, extract cell-free nucleic acids, and purify cell-free nucleic acids. In some instances, devices, systems and kits disclosed herein comprise components to obtain a sample, extract cell-free nucleic acids, purify cell-free nucleic acids, and prepare a library of the cell-free nucleic acids. A kit can comprise components to obtain a sample, extract cell-free nucleic acids, purify cell-free nucleic acids, and sequence cell-free nucleic acids. A kit can comprise components to obtain a sample, extract cell-free nucleic acids, purify cell-free nucleic acids, prepare a library of the cell-free nucleic acids, and sequence the cell-free nucleic acids. Components for obtaining a sample can be a transdermal puncture device and a filter for obtaining plasma from blood. Components for extracting and purifying cell-free nucleic acids can comprise buffers, beads and magnets. Buffers, beads and magnets may be supplied at volumes appropriate for receiving a general sample volume from a finger prick (e.g., 50-150 μl of blood).

A kit can comprise a receptacle for receiving the biological sample. The receptacle may be configured to hold a volume of a biological sample between 1 μl and 1 ml. The receptacle may be configured to hold a volume of a biological sample between 1 μl and 500 μl. The receptacle may be configured to hold a volume of a biological sample between 1 μl and 200 μl. The receptacle may have a defined volume that is the same as a suitable volume of sample for processing and analysis by the rest of the device/system components. In some instances, devices, systems and kits do not comprise a receptacle for receiving the biological sample. In some instances, the sample purifier receives the biological sample directly. The sample purifier may have a defined volume that is suitable for processing and analysis by the rest of the device/system components. The user can preserve or send the analyzed sample to another location (e.g., lab, clinic) for additional analysis or confirmation of results obtained at a point of care. The kit may be used to separate plasma from blood. The plasma may be analyzed at point of care and the cells from the blood shipped to another location for analysis. A kit can comprise a transport compartment or storage compartment for these purposes. The transport compartment or storage compartment may be capable of containing a biological sample, a component thereof, or a portion thereof. The transport compartment or storage compartment may be capable of containing the biological sample, portion thereof, or component thereof, during transit to a site remote to the immediate user. The transport compartment or storage compartment may be capable of containing cells that are removed from a biological sample, so that the cells can be sent to a site remote to the immediate user for testing. Non-limiting examples of a site remote to the immediate user may be a laboratory or a clinic when the immediate user is at home. In some instances, the home does not have a machine or additional device to perform an additional analysis of the biological sample. The transport compartment or storage compartment may be capable of containing a product of a reaction or process that result from adding the biological sample to the device. In some instances, the product of the reaction or process is a biological sample component bound to a binding moiety described herein. In some instances, the transport compartment or storage compartment comprises an absorption pad, a paper, a glass container, a plastic container, a polymer matrix, a liquid solution, a gel, a preservative, or a combination thereof.

The transport compartment or storage compartment can comprise a preservative. The preservative may also be referred to herein as a stabilizer or biological stabilizer. In some instances, the device, system or kit comprises a preservative that reduces enzymatic activity during storage and/or transportation. In some instances, the preservative is a whole blood preservative. Non-limiting examples of whole blood preservatives, or components thereof, are glucose, adenine, citric acid, trisodium citrate, dextrose, sodium di-phosphate, and monobasic sodium phosphate. In some instances, the preservative comprises EDTA. EDTA may reduce enzymatic activity that would otherwise degrade nucleic acids. In some instances, the preservative comprises formaldehyde. In some instances, the preservative is a known derivative of formaldehyde. Formaldehyde, or a derivative thereof, may cross link proteins and therefore stabilize cells and prevent cell lysis.

A kit can comprise a sample collector. In some instances, the sample collector is provided separately from the rest of the kit. In some instances, the sample collector is integrated with a receptacle described herein. In some instances, the sample collector may be a cup, tube, capillary, or well for applying the biological fluid. In some instances, the biological fluid collected is whole blood. In some instances, the whole blood is collected from the subject by venipuncture. In some instances, the whole blood is collected from finger stick that provides a capillary blood sample. In some instances, the sample collector may be a cup for providing urine. In some instances, the sample collector may comprise a pipette for providing urine. In some instances, the sample collector may be a capillary integrated with a device disclosed herein for applying blood. In some instances, the sample collector may be a plasma separation device. In some instances, the sample collector may be dried blood spot card. In some instances, the dried blood spot card may comprise a plasma separation card for collecting venous or capillary blood spots and separating plasma samples from the dried blood spots. In some instances, the sample collector may be tube, well, pad or paper integrated with a device disclosed herein for applying saliva. In some instances, the sample collector may be pad or paper for applying sweat.

A kit can comprise a transdermal puncture device. Non-limiting examples of transdermal puncture devices are needles and lancets. In some instances, the sample collector comprises the transdermal puncture device. A kit can comprise a microneedle, microneedle array or microneedle patch. A kit can comprise a hollow microneedle. By way of non-limiting example, the transdermal puncture device is integrated with a well or capillary so that as the subject punctures their finger, blood is released into the well or capillary where it will be available to the system or device for analysis of its components. In some instances, the transdermal puncture device is a push button device with a needle or lancet in a concave surface. In some instances, the needle is a microneedle. In some instances, the transdermal puncture device comprises an array of microneedles. By pressing an actuator, button or location on the non-needle side of the concave surface, the needle punctures the skin of the subject in a more controlled manner than a lancet. Furthermore, the push button device may comprise a vacuum source or plunger to help draw blood from the puncture site.

A kit can comprise a sample processor, wherein the sample processor modifies a biological sample to remove a component of the sample or separate the sample into multiple fractions (e.g., blood cell fraction and plasma or serum). The sample processor may comprise a sample purifier, wherein the sample purifier is configured to remove an unwanted substance or non-target component of a biological sample, thereby modifying the sample. Depending on the source of the biological sample, unwanted substances can include, but are not limited to, proteins (e.g., antibodies, hormones, enzymes, serum albumin, lipoproteins), free amino acids and other metabolites, microvesicles, nucleic acids, lipids, electrolytes, urea, urobilin, pharmaceutical drugs, mucous, bacteria, and other microorganisms, and combinations thereof. In some instances, the sample purifier separates components of a biological sample disclosed herein. In some instances, sample purifiers disclosed herein remove components of a sample that would inhibit, interfere with or otherwise be detrimental to the later process steps such as detection. In some instances, the resulting modified sample is enriched for target analytes.

In some instances, the sample purifier comprises a separation material for removing unwanted substances other than patient cells from the biological sample. Useful separation materials may include specific binding moieties that bind to or associate with the substance. Binding can be covalent or noncovalent. In some instances, a sample purifier disclosed herein comprises a binding moiety that binds a nucleic acid, protein, cell surface marker, or microvesicle surface marker in the biological sample. In some instances, the binding moiety comprises an antibody, antigen binding antibody fragment, a ligand, a receptor, a peptide, a small molecule, or a combination thereof.

In some instances, sample purifiers disclosed herein comprise a filter. In some instances, sample purifiers disclosed herein comprise a membrane. The filter or membrane is capable of separating or removing cells, cell particles, cell fragments, blood components other than cell-free nucleic acids, or a combination thereof, from the biological samples disclosed herein.

In some instances, the sample purifier facilitates separation of plasma or serum from cellular components of a blood sample. In some instances, the sample purifier facilitates separation of plasma or serum from cellular components of a blood sample before starting a sequencing reaction. Plasma or serum separation can be achieved by several different methods such as centrifugation, sedimentation or filtration. In some instances, the sample purifier comprises a filter matrix for receiving whole blood, the filter matrix having a pore size that is prohibitive for cells to pass through, while plasma or serum can pass through the filter matrix uninhibited. In some instances, the filter matrix combines a large pore size at the top with a small pore size at the bottom of the filter, which leads to very gentle treatment of the cells preventing cell degradation or lysis, during the filtration process. This is advantageous because cell degradation or lysis would result in release of nucleic acids from blood cells or maternal cells that can contaminate target cell-free nucleic acids.

A kit can comprise vertical filtration, driven by capillary force to separate a component or fraction from a sample (e.g., plasma from blood). The sample purifier may comprise a lateral filter (e.g., sample does not move in a gravitational direction or the sample moves perpendicular to a gravitational direction). The sample purifier may comprise a vertical filter (e.g., sample moves in a gravitational direction). The sample purifier may comprise a vertical filter and a lateral filter. The sample purifier may be configured to receive a sample or portion thereof with a vertical filter, followed by a lateral filter. The sample purifier may be configured to receive a sample or portion thereof with a lateral filter, followed by a vertical filter. In some instances, a vertical filter comprises a filter matrix. In some instances, the filter matrix of the vertical filter comprises a pore with a pore size that is prohibitive for cells to pass through, while plasma can pass the filter matrix uninhibited. In some instances, the filter matrix comprises a membrane that is especially suited for this application because it combines a large pore size at the top with a small pore size at the bottom of the filter, which leads to very gentle treatment of the cells preventing cell degradation during the filtration process.

In some instances, the sample purifier comprises an appropriate separation material, e.g., a filter or membrane, that removes unwanted substances from a biological sample without removing cell-free nucleic acids. In some embodiments wherein the biological sample is whole blood, standard collection techniques using centrifugation of whole blood are used in the preparation of the cfDNA enriched biological sample. In some instances, the separation material separates substances in the biological sample based on size, for example, the separation material has a pore size that excludes a cell but is permeable to cell-free nucleic acids. Therefore, when the biological sample is blood, the plasma or serum can move more rapidly than a blood cell through the separation material in the sample purifier, and the plasma or serum containing any cell-free nucleic acids can permeate the holes of the separation material. In some instances, the biological sample is blood, and the cell that is slowed and/or trapped in the separation material is a red blood cell, a white blood cell, or a platelet. In some instances, the cell is from a tissue that contacted the biological sample in the body, including, but not limited to, a bladder or urinary tract epithelial cell (in urine), or a buccal cell (in saliva). In some instances, the cell is a bacterium or other microorganism.

In some instances, the sample purifier is capable of slowing and/or trapping a cell without damaging the cell, thereby avoiding the release of cell contents including cellular nucleic acids and other proteins or cell fragments that could interfere with subsequent evaluation of the cell-free nucleic acids. This can be accomplished, for example, by a gradual, progressive reduction in pore size along the path of a lateral flow strip or other suitable assay format, to allow gentle slowing of cell movement, and thereby minimize the force on the cell. In some instances, at least 95%, at least 98%, at least 99%, or up to 100% of the cells in a biological sample remain intact when trapped in the separation material. In addition to or independently of size separation, the separation material can trap or separate unwanted substances based on a cell property other than size, for example, the separation material can comprise a binding moiety that binds to a cell surface marker. In some instances, the binding moiety is an antibody or antigen binding antibody fragment. In some instances, the binding moiety is a ligand or receptor binding protein for a receptor on a blood cell or microvesicle.

A kit can comprise a separation material that moves, draws, pushes, or pulls the biological sample through the sample purifier, filter and/or membrane. In some instances, the material is a wicking material. Examples of appropriate separation materials used in the sample purifier to remove cells include, but are not limited to, polyvinylidene difluoride, polytetrafluoroethylene, acetylcellulose, nitrocellulose, polycarbonate, polyethylene terephthalate, polyethylene, polypropylene, glass fiber, borosilicate, vinyl chloride, silver. Suitable separation materials may be characterized as preventing passage of cells. In some instances, the separation material is not limited as long as it has a property that can prevent passage of the red blood cells. In some instances, the separation material is a hydrophobic filter, for example a glass fiber filter, a composite filter, for example Cytosep (e.g., Ahlstrom Filtration or Pall Specialty Materials, Port Washington, NY), or a hydrophilic filter, for example cellulose (e.g., Pall Specialty Materials). In some instances, whole blood can be fractionated into red blood cells, white blood cells and serum components for further processing according to the methods of the present disclosure using a commercially available kit (e.g., Arrayit Blood Card Serum Isolation Kit, Cat. ABCS, Arrayit Corporation, Sunnyvale, CA).

In some instances, the sample purifier comprises at least one filter or at least one membrane characterized by at least one pore size. In some instances, the sample purifier comprises multiple filters and/or membranes, wherein the pore size of at least a first filter or membrane differs from a second filter or membrane. In some instances, at least one pore size of at least one filter/membrane is about 0.05 microns to about 10 microns. In some instances, the pore size is about 0.05 microns to about 8 microns. In some instances, the pore size is about 0.05 microns to about 6 microns. In some instances, the pore size is about 0.05 microns to about 4 microns. In some instances, the pore size is about 0.05 microns to about 2 microns. In some instances, the pore size is about 0.05 microns to about 1 micron. In some instances, at least one pore size of at least one filter/membrane is about 0.1 microns to about 10 microns. In some instances, the pore size is about 0.1 microns to about 8 microns. In some instances, the pore size is about 0.1 microns to about 6 microns. In some instances, the pore size is about 0.1 microns to about 4 microns. In some instances, the pore size is about 0.1 microns to about 2 microns. In some instances, the pore size is about 0.1 microns to about 1 micron.

In some instances, the sample purifier is characterized as a gentle sample purifier. Gentle sample purifiers, such as those comprising a filter matrix, a vertical filter, a wicking material, or a membrane with pores that do not allow passage of cells, are particularly useful for analyzing cell-free nucleic acids.

In some instances, the sample processor is configured to separate blood cells from whole blood. In some instances, the sample processor is configured to isolate plasma from whole blood. In some instances, the sample processor is configured to isolate serum from whole blood. In some instances, the sample processor is configured to isolate plasma or serum from less than 1 milliliter of whole blood. In some instances, the sample processor is configured to isolate plasma or serum from less than 1 milliliter of whole blood. In some instances, the sample processor is configured to isolate plasma or serum from less than 500 μL of whole blood. In some instances, the sample processor is configured to isolate plasma or serum from less than 400 μL of whole blood. In some instances, the sample processor is configured to isolate plasma or serum from less than 300 μL of whole blood. In some instances, the sample processor is configured to isolate plasma or serum from less than 200 μL of whole blood. In some instances, the sample processor is configured to isolate plasma or serum from less than 150 μL of whole blood. In some instances, the sample processor is configured to isolate plasma or serum from less than 100 μL of whole blood.

A kit can comprise a binding moiety for producing a modified sample depleted of cells, cell fragments, nucleic acids or proteins that are unwanted or of no interest. A kit can comprise a binding moiety for reducing cells, cell fragments, nucleic acids or proteins that are unwanted or of no interest, in a biological sample. A kit can comprise a binding moiety for producing a modified sample enriched with target cell, target cell fragments, target nucleic acids or target proteins.

A kit can comprise a binding moiety capable of binding a nucleic acid, a protein, a peptide, a cell surface marker, or microvesicle surface marker. A kit can comprise a binding moiety for capturing an extracellular vesicle or extracellular microparticle in the biological sample. In some instances, the extracellular vesicle contains at least one of DNA and RNA. A kit can comprise reagents or components for analyzing DNA or RNA contained in the extracellular vesicle. In some instances, the binding moiety comprises an antibody, antigen binding antibody fragment, a ligand, a receptor, a protein, a peptide, a small molecule, or a combination thereof.

A kit can comprise a binding moiety capable of interacting with or capturing an extracellular vesicle that is released from a cell. In some instances, the extracellular vesicle is released from an organ, gland or tissue. By way of non-limiting example, the organ, gland or tissue may be diseased, aging, infected, or growing. Non-limiting examples of organs, glands and tissues are brain, liver, heart, kidney, colon, pancreas, muscle, adipose, thyroid, prostate, breast tissue, and bone marrow. A kit can be configured for capable of capturing and discarding an extracellular vesicle or extracellular microparticle from a maternal sample to enrich the sample for cell free nucleic acids.

In some instances, the binding moiety is attached to a solid support, wherein the solid support can be separated from the rest of the biological sample or the biological sample can be separated from the solid support, after the binding moiety has made contact with the biological sample. Non-limiting examples of solid supports include a bead, a nanoparticle, a magnetic particle, a chip, a microchip, a fibrous strip, a polymer strip, a membrane, a matrix, a column, a plate, or a combination thereof.

A kit can comprise a cell lysis reagent. Non-limiting examples of cell lysis reagents include detergents such as NP-40, sodium dodecyl sulfate, and salt solutions comprising ammonium, chloride, or potassium. A kit can comprise a cell lysis component. The cell lysis component may be structural or mechanical and capable of lysing a cell. By way of non-limiting example, the cell lysis component may shear the cells to release intracellular components such as nucleic acids. In some embodiments, a kit does not comprise a cell lysis reagent.

### Computer Systems

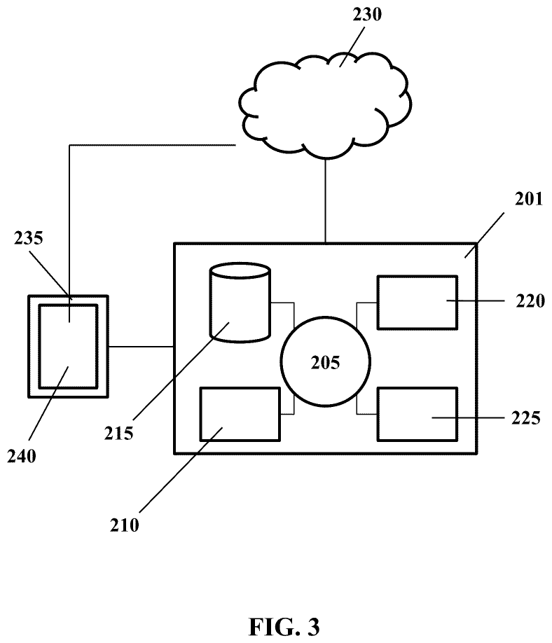

The present disclosure provides computer systems that are programmed to implement methods of the disclosure. FIG. 3 shows a computer system 301 that is programmed or otherwise configured to, for example, quantify the amount of minority component in a sample based on genomic data, identify a plurality of minority components present in the sequenced cfDNA enriched biological sample, assign a designation that represents a low-confidence estimate of minor variant frequency to individual identified minority components present in the sequenced cfDNA enriched biological sample, and/or average a plurality of low-confidence estimates of minor variant frequency across a plurality of sequenced genomic loci to produce an estimation of minority component frequency in the cfDNA enriched biological sample.

The computer system 301 may regulate various aspects of analysis, calculation, and generation of the present disclosure, such as, for example, quantifying the amount of minority component in a sample based on genomic data, identifying a plurality of minority components present in the sequenced cfDNA enriched biological sample, assigning a designation that represents a low-confidence estimate of minor variant frequency to individual identified minority components present in the sequenced cfDNA enriched biological sample, and/or averaging a plurality of low-confidence estimates of minor variant frequency across a plurality of sequenced genomic loci to produce an estimation of minority component frequency in the cfDNA enriched biological sample. The computer system 301 may be an electronic device of a user or a computer system that is remotely located with respect to the electronic device. The electronic device may be a mobile electronic device.

The computer system 301 includes a central processing unit (CPU, also “processor” and “computer processor” herein) 305, which may be a single core or multi core processor, or a plurality of processors for parallel processing. The computer system 301 also includes memory or memory location 310 (e.g., random-access memory, read-only memory, flash memory), electronic storage unit 315 (e.g., hard disk), communication interface 320 (e.g., network adapter) for communicating with one or more other systems, and peripheral devices 325, such as cache, other memory, data storage and/or electronic display adapters. The memory 310, storage unit 315, interface 320 and peripheral devices 325 are in communication with the CPU 305 through a communication bus (solid lines), such as a motherboard. The storage unit 315 may be a data storage unit (or data repository) for storing data. The computer system 301 may be operatively coupled to a computer network (“network”) 330 with the aid of the communication interface 320. The network 330 may be the Internet, an internet and/or extranet, or an intranet and/or extranet that is in communication with the Internet.

The network 330 in some cases is a telecommunication and/or data network. The network 330 may include one or more computer servers, which may enable distributed computing, such as cloud computing. For example, one or more computer servers may enable cloud computing over the network 330 (“the cloud”) to perform various aspects of analysis, calculation, and generation of the present disclosure, such as, for example, quantifying the amount of minority component in a sample based on genomic data, identifying a plurality of minority components present in the sequenced cfDNA enriched biological sample, assigning a designation that represents a low-confidence estimate of minor variant frequency to individual identified minority components present in the sequenced cfDNA enriched biological sample, and/or averaging a plurality of low-confidence estimates of minor variant frequency across a plurality of sequenced genomic loci to produce an estimation of minority component frequency in the cfDNA enriched biological sample. Such cloud computing may be provided by cloud computing platforms such as, for example, Amazon Web Services (AWS), Microsoft Azure, Google Cloud Platform, and IBM cloud. The network 330, in some cases with the aid of the computer system 301, may implement a peer-to-peer network, which may enable devices coupled to the computer system 301 to behave as a client or a server.

The CPU 305 may comprise one or more computer processors and/or one or more graphics processing units (GPUs). The CPU 305 may execute a sequence of machine-readable instructions, which may be embodied in a program or software. The instructions may be stored in a memory location, such as the memory 310. The instructions may be directed to the CPU 305, which may subsequently program or otherwise configure the CPU 305 to implement methods of the present disclosure. Examples of operations performed by the CPU 305 may include fetch, decode, execute, and writeback.

The CPU 305 may be part of a circuit, such as an integrated circuit. One or more other components of the system 301 may be included in the circuit. In some cases, the circuit is an application specific integrated circuit (ASIC).

The storage unit 315 may store files, such as drivers, libraries and saved programs. The storage unit 315 may store user data, e.g., user preferences and user programs. The computer system 301 in some cases may include one or more additional data storage units that are external to the computer system 301, such as located on a remote server that is in communication with the computer system 301 through an intranet or the Internet.

The computer system 301 may communicate with one or more remote computer systems through the network 330. For instance, the computer system 301 may communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user may access the computer system 301 via the network 330.

Methods as described herein may be implemented by way of machine (e.g., computer processor) executable code stored on an electronic storage location of the computer system 301, such as, for example, on the memory 310 or electronic storage unit 315. The machine executable or machine readable code may be provided in the form of software. During use, the code may be executed by the processor 305. In some cases, the code may be retrieved from the storage unit 315 and stored on the memory 310 for ready access by the processor 305. In some situations, the electronic storage unit 315 may be precluded, and machine-executable instructions are stored on memory 310.

The code may be pre-compiled and configured for use with a machine having a processer adapted to execute the code, or may be compiled during runtime. The code may be supplied in a programming language that may be selected to enable the code to execute in a pre-compiled or as-compiled fashion.

Aspects of the systems and methods provided herein, such as the computer system 301, may be embodied in programming. Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media may include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 301 may include or be in communication with an electronic display 335 that comprises a user interface (UI) 340 for quantifying the amount of minority component in a sample based on genomic data, identifying a plurality of minority components present in the sequenced cfDNA enriched biological sample, assigning a designation that represents a low-confidence estimate of minor variant frequency to individual identified minority components present in the sequenced cfDNA enriched biological sample, and/or averaging a plurality of low-confidence estimates of minor variant frequency across a plurality of sequenced genomic loci to produce an estimation of minority component frequency in the cfDNA enriched biological sample. Examples of UIs include, without limitation, a graphical user interface (GUI) and web-based user interface.

Methods and systems of the present disclosure may be implemented by way of one or more algorithms. An algorithm may be implemented by way of software upon execution by the central processing unit 305. The algorithm can, for example, quantify the amount of minority component in a sample based on genomic data.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1: Low-Coverage, Genome-Wide Identification of Minority cfDNA Contributors

### Theoretical Background:

The dbSNP database contains approximately 11.4 million SNPs with a minor allele frequency greater than 20% in human populations. Based on Hardy-Weinberg equilibrium expectations, at least 500,000 SNPs should be alternate homozygotes when comparing two random individuals. Using a low-coverage whole-genome sequencing of 0.1× genomic coverage (about 1 in 10 bases of the genome sequenced), it is expected that about 50,000 of these SNP loci of alternative homozygous genotypes between two unrelated individuals will have some level of sequencing coverage in a typical sample. If a minor constituent of cfDNA is present at 0.1% rate, then approximately 1 in 1000 molecules is expected to be derived from the minor contributor, which also approximates 50 alternate homozygote SNP loci. 50 events are expected to be sufficient to adequately estimate the presence of minor cfDNA components versus background noise.

Heterozygous and homozygous sites can similarly be incorporated to improve both the number of sites sampled and, consequently, the sensitivity of the assay. The method detects SNP alleles that are different from the majority cfDNA set and not explicitly linked to the SNP alleles known (or not) to be present in the minority cfDNA set. As such, a high confidence genotyping of the reference (majority component) may strongly improve confidence. But the same is not required for a reference of the minority component.

Empirical Estimation of Minority cfDNA Quantitation:

Mixtures of plasma were prepared from two individuals, one male and one female, ranging from 100% female to 0.05% female, and sequenced at approximately 0.2× genomic coverage. In addition, unmixed references for each sample were prepared and sequenced at approximately 10× genomic coverage to identify potential SNP sites. In the mixtures, the relative abundance of the Y-chromosome was used to calibrate the empirically observed minor component frequency in this set.

Using the structure outlined below, SNP sites were identified and screened in mixtures to assess the ability to quantitatively assess minor cfDNA levels down to 0.1% presence. These data support the capacity to identify minor cfDNA levels to at least 0.1%, and possibly 0.05%.
1. Identify homozygous SNP loci in the recipient (major component) subject.
2. Optionally, identify heterozygous and alternate homozygous SNP loci in the donor (minor component) subject.
3. Determine frequency of non-recipient genotypes at pre-screened recipient SNP loci in mixtures.
4. Compare resulting frequencies with known mixture levels.

### Example 2: Transplant Rejection Monitoring

This example provides a strategy for monitoring changes over time in the frequency of minor components of cfDNA derived from transplanted tissue. The screening tests can be performed from capillary based collections on microliter volumes of plasma.

An increasing presence of transplant-derived cfDNA over time can indicate of organ failure and/or rejection. Using this method to screen SNP loci that differ between donor and recipient, an estimation of donor cfDNA amount may be determined. Should this value rise over time, the patient may be notified to follow up with physicians for further care. The methods described herein may be used to tune individual background of the subject to the testing format to create personalized profiling. Longitudinal sampling over a period of time allows for monitoring of transplant rejection and transplant organ failure. In some embodiments, the frequency of longitudinal sampling is faster (e.g., several samples taken from the subject over a period of days or weeks) as opposed to a slower frequency of sampling (e.g., monthly, bi-monthly, quarterly, bi-annually, or annually). In some embodiments, a faster frequency of longitudinal sampling is used when the subject presents with an acute condition. In some embodiments, a faster frequency of longitudinal sampling is used when the subject is at elevated risk for transplant rejection. In some embodiments, a faster frequency of longitudinal sampling is used when the subject is at elevated risk for transplant organ failure. In some embodiments, a slower frequency of longitudinal sampling is used when the subject is has a stable condition. In some embodiments, a slower frequency of longitudinal sampling is used to monitor for changes within a stable condition in the subject. The estimation of minor component frequency of dd-cfDNA may further be evaluated to determine any changes in detected percentage of dd-cfDNA over host-derived-cfDNA across a plurality of samples by including analysis comprising data partitioning. During the analysis, partitioning sequencing reads of the biological sample into a set of contaminant sequencing reads and a set of target sequencing reads can aid in detection accuracy, sensitivity and/or specificity of transplant rejection monitoring or transplant failure monitoring. By partitioning the set of contaminating sequencing reads and the set of target sequencing reads, a subject may be classified and/or characterized with a significant increase to detection accuracy, sensitivity and/or specificity of the detecting and/or determining a presence of the set of target sequencing reads and thus improve a classification and/or characterization of the subject with at least the set of target sequencing reads.

### Example 3: Oncology Screening

This example provides a strategy for monitoring changes over time in the frequency of minor components of cfDNA derived from tumor samples. The screening tests can be performed from capillary based collections on microliter volumes of plasma.

In the case of oncology screening, the minor components of cfDNA to be identified would be tumor-derived. As such, this use case differs from some others in that, rather than screening for a mixture of two sets of germline SNP variants, here the objective would be to identify a significant increase in somatic variants over time; such variants number in the tens to hundreds per megabase of tumor tissue and thus provide a large number of potential targets. Nevertheless, the assumptions described above should still apply in such use cases although using a different reference approach (somatic variant sets versus germline SNPs). In some versions, this use case may be informed by past patient history (e.g. tumor biopsy data, etc.), but this is not a required element of the screening.

The method is used to detect an increase in somatic mutations in a minority component of cfDNA comprising ctDNA. The increase in somatic mutations can be due to inefficient DNA damage repair in pre-malignant and malignant cells and represent a driving force in cancer progression. Identification of an increase of tumor cells unable to efficiently repair DNA damage which leads to an increase in somatic mutations in ctDNA enables: i) earlier initiation of a treatment in the subject; ii) earlier modification of an ongoing treatment in the subject, iii) an earlier gating decision for treatment switching in the subject, or iv) any combination thereof.

The method is used to detect an increase in clonal somatic mutations in a minority component of cfDNA comprising ctDNA. An increase in clonal somatic mutations in later-taken sample compared to an earlier-taken sample from the subject can indicate a progression of a cancer or a metastasis of a cancer in the subject as a higher percentage of clonal somatic mutations can be derived from a higher proportion of ctDNA in the total cfDNA sample. Identification of an increase in clonal somatic mutations in later-taken sample enables: i) earlier initiation of a treatment in the subject; ii) earlier modification of an ongoing treatment in the subject, iii) an earlier gating decision for treatment switching in the subject, or iv) any combination thereof.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

1. A method for analyzing cell free DNA (cfDNA), the method comprising:
a) obtaining a biological sample derived from a subject, wherein the biological sample comprises cfDNA;
b) enriching a proportion of cfDNA within the biological sample;
c) sequencing the cfDNA enriched biological sample using low-coverage, genome-wide nucleic acid sequencing;
d) identifying a plurality of minority components present in the sequenced cfDNA enriched biological sample;
e) assigning a designation that represents a low-confidence estimate of minor variant frequency to individual identified minority components present in the sequenced cfDNA enriched biological sample; and
f) averaging a plurality of low-confidence estimates of minor variant frequency across a plurality of sequenced genomic loci to produce an estimation of minority component frequency in the cfDNA enriched biological sample.

2. The method of claim 1, wherein the designation in e) is a binary classifier for the individual identified minority components to distinguish a plurality of sequenced genomic loci from sequenced genomic loci not identified as having a minor variant in the sequenced cfDNA enriched biological sample.

3. The method of claim 1, wherein the identifying in d) comprises:
i. aligning raw sequence data generated with low-coverage whole genome sequencing (lcWGS) in c) to a reference sequence;
ii. marking duplicate reads of sequenced fragments;
iii. conducting pre-processing of BAM files generated following lcWGS by base quality score recalibration (BQSR);
iv. performing local realignment of sequences from pre-processed BAM files to produce analysis-ready BAM files; and
v. performing variant calling on analysis-ready BAM files to identify the minority components.

4. The method of claim 1, wherein a reference sample comprising genomic DNA derived from the subject is analyzed to distinguish somatic genotypes present in the subject from identified minority components in the cfDNA enriched biological sample.

5. The method of claim 4, wherein the somatic genotypes are identified through high confidence genotyping.

6. (canceled)

7. The method of claim 1, wherein the sites sequenced with low-coverage, genome-wide nucleic acid sequencing are agnostic to pre-defined genomic loci.

8. The method of claim 1, wherein the estimation of minority component frequency in the cfDNA enriched biological sample is a quantitative detection of a minority component present in the cfDNA of the biological sample.

9.-15. (canceled)

16. The method of claim 1, wherein the individual identified minority components in cfDNA in e) comprise alternate heterozygous alleles or alternate homozygous alleles when compared to the alleles of genomic DNA from the subject.

17. (canceled)

18. The method of claim 1, wherein the variants detected comprise single-nucleotide polymorphisms (SNPs), small insertions or deletions (INDELs), variable number of tandem repeats (VNTR), simple sequence repeats (SSR), simple tandem repeats (STR), or any combination thereof.

19.-22. (canceled)

23. The method of claim 16, wherein the alternate heterozygous alleles or alternate homozygous alleles are derived from cfDNA from pre-malignant or malignant cells of the subject, derived from cfDNA from one or more infectious agents residing within the subject, or derived from a donor subject.

24. (canceled)

25. (canceled)

26. The method of claim 23, wherein the donor subject is an embryo or a fetus.

27. (canceled)

28. The method of claim 23, wherein the donor subject has provided a tissue or an organ transplant into the subject which serves as a host.

29. The method of claim 1, wherein the estimation of minority component frequency in the cfDNA enriched biological sample in f) is used for transplantation monitoring, or for oncology detection or oncology monitoring.

30.-42. (canceled)

43. The method of claim 23, comprising monitoring progression of an infectious disease.

44.-46. (canceled)

47. The method of claim 23, wherein a development or progression of pregnancy complications is monitored.

48. The method of claim 1, further comprising analyzing fragment patterning in the cfDNA enriched biological sample.

49. The method of claim 1, further comprising imputing missing SNP genotypes in the sequenced cfDNA enriched biological sample.

50. The method of claim 1, further comprising calculating and evaluating regional linkage disequilibrium ratios between variant alleles detected to enhance the calculated estimation of minority component frequency in the cfDNA enriched biological sample.

51. The method of claim 1, further comprising individual subject level tuning comprising longitudinal sampling of biological samples obtained from the subject to screen for change in minor cfDNA components over time.

52. The method of claim 1, wherein the low-coverage, genome-wide nucleic acid sequencing is unbiased sequencing.

53. (canceled)

54. (canceled)
